(19) United States
(12) Patent Application Publication
(10) Pub. No.: US 2010/0035249 A1
(43) Pub. Date: Feb. 11, 2010

(54) RNA SEQUENCING AND ANALYSIS USING SOLID SUPPORT

(75) Inventors: Yoshihide Hayashizaki, Yokohama-shi (JP); Piero Carninci, Yokohama-shi (JP); Mutsumi Katayama, Yokohama-shi (JP); Shintaro Katayama, Yokohama-shi (JP); Masayoshi Itoh, Yokohama-shi (JP); Matthias Harbers, Yokohama-shi (JP)

Correspondence Address: FOLEY AND LARDNER LLP;SUITE 500, 3000 K STREET NW, WASHINGTON, DC, 20007, US

(73) Assignees: KABUSHIKI KAISHA DNAFORM; RIKEN

(21) Appl. No.: 12/186,009

(22) Filed: Aug. 5, 2008

(51) Int. Cl. C12Q 1/68

(52) U.S. Cl. 435/6

(57) ABSTRACT

The present invention provides methods for the sequencing of all RNA species within an RNA sample, such as the RNA content obtained from a cell, a tissue, a living organism, or from an artificial source. RNA molecules within the samples are labeled in a RNA-specific manner prior to immobilization on a solid support. One label is used to mark the location of the RNA molecule on the solid support, whereas the second label is used to mark selectively the S′ end of full-length mRNA molecules. RNA molecules are sequenced while being bound to the solid support in one or more sequencing reactions, and sequences of individual RNA molecules can be forwarded to computational analysis for assembling sequence information from individual sequencing reads obtained from the same location on the solid support. Not only unsupervised expression profiling on a genome-wide scale, but also the direct analysis of RNA-RNA interactions become possible as revealed by the analysis of the sequencing information obtained along with genomic information.

### FIELD OF THE INVENTION

The present invention relates to molecular biology methods for the analysis of RNA molecules within a cell or in biological samples by means of obtaining sequence information from individual RNA molecules. Such sequence information may be obtained randomly, from one end or both ends of an RNA molecule, or from the entire sequence of an RNA molecule. Moreover, the invention relates to the analysis of such sequence information and search for RNA molecules that could interact with each other.

### BACKGROUND ART

Driven by the success of the human genome project and an interest in obtaining large amounts of genomic sequence information for diagnostic purpose, whole genome sequencing has entered into a new period with the availability of next-generation sequencing technologies [Mardis E. R., Trends in Genetics 24, 133-141 (2008), von Bubnoff A., Cell 132, 721-723 (2008)], Next-generation sequencing no longer looks at individual DNA or RNA molecules, but rather targets at the parallel sequencing of a very large number of DNA or RNA fragments. Present approaches in such next-generation sequencing commonly achieve this goal by immobilizing one reaction component on a solid support to monitor the primer extension reaction of one DNA or RNA strand along the sequencing template. Monitoring the extension reaction of one molecule or a group of molecules having the same sequence information in a given location defines the sequence output for each template. With an increasing number of sequencing reactions performed on the same solid support and growing numbers for incorporation reactions that can be detected for each primer extension reaction, large amounts of sequence information can be obtained, greatly exceeding the output of classical capillary sequencing [refer to Sambrook J. and Russuell D. W., Molecular Cloning, A Laboratory Manual, Cold Spring Harbor Laboratory Press, New York, 2001, and Sensen C. W., Essentials of Genomics and Bioinformatics, Wiley-VCH, Weinheim 2002, page 165, for information on classical sequencing approaches].

Of particular interest for the next-generation sequencing are novel approaches for the detection and sequencing of single DNA or RNA molecules as recently reviewed in Metzker M. L., Genome Res. 15, 1767-1776 (2005), Kling J., Nature Biotechnology 23, 1333-1335 (2005), Shendure J. et al., Nature Review Genetics 5, 335-344 (2004), Mardis E. R., Trends in Genetics 24, 133-141 (2008), and von Bubnoff A., Cell 132, 721′-723 (2008). Some of those approaches are subject to commercial applications as offered by companies that have developed special instruments and reagent kits for high-throughput next-generation sequencing. For example, the GS FLX sequencer from 454 Life Sciences now part of ROCHE Diagnostics [http://www.454.com/], the Genome Analyzer from Illumina, formerly Solexa [http://www.illumina.com], the SOLID System from Applied Biosystems [http://marketing.appliedbiosystems.com/mk/get/SOLID-KNOWLEDGE_LANDING], and the HeliScope™ Single Molecule Sequencer from Helicos BioScience [http://www.helicosbio.com/ are presently offered on the market. Other companies are actively working on similar or even more powerful devices, such as: Pacific Biosciences http://www.pacificbiosciences.com/index.php], Visigen [http://www.visigenbio.com/index.html], or GeneoVoxx [http://www.genovoxx.de/].

Templates for the next-generation sequencing are commonly obtained from genomic DNA that is fragmentized to yield DNA molecules suitable in length for conducting the sequencing reaction. After fragmentation, the ends of such DNA fragments are modified to introduce priming sites of known sequence. Those priming sites can be used in amplification reactions to generate groups of DNA molecules having the same sequence and containing regions for annealing a sequencing primer prior to initiating the sequencing reaction and to introduce functional groups for binding to a solid support. It is desirable to restrict the number of manipulations to a minimum to avoid a loss of materials or any bias that may occur by low or uneven yields in the modification and amplification reactions. Most of the devices still require an amplification step, though the approaches of Helicos and Pacific BioScience, for example, are targeting at the detection of each single molecule rather than groups of molecules having the same sequence, thus avoiding the need for template amplification. Single molecule detection can greatly increase the throughput of the sequencer while at the same time reducing the number of manipulations required for template preparation. However, even for single molecule detection the template must have a proper priming site to drive sequencing in a polymerase reaction and/or modifications for binding the template to a solid support. The use of inherited features found in naturally occurring RNA species may allow reducing the need for such modification steps.

While different protocols for template preparation from genomic DNA are well established, preparing sequencing templates from RNA still provides different challenges, thus far not addressed by a single, unified approach for monitoring all RNA molecules present in a biological sample. At this moment, rather distinct protocols have been developed for cloning, detecting and/or monitoring the expression of specific RNA species. In particular, a focus has been placed on distinct approaches to mRNA and short RNA cloning and sequencing [refer to Harbers, M. Genomics 91, 232-242 (2008) on present cloning approaches]. Processing and sequencing of mRNA molecules provides onerous challenges due to uneven distribution of different mRNA molecules within samples and the presence of very long mRNA molecules. Most protocols require an amplification step during sample preparation that can lead to an uneven representation of different RNA molecules within the sequencing sample. Due to bias amplification reactions explicit reductions in the representation of very long mRNA molecules have been reported in the literature [Karrer E. E., et al., Proc Natl Acad Sci USA 92, 3814-3818 (1995)]. This has motivated the development of approaches using fragments of limited length in the amplification reactions [Chen J. and Rattray M., BMC Genomics. 7, doi:10.1186/1471-2164-7-77 (2006)]. Fragmentation of mRNA or cDNA molecules derived thereof has to consider from which portion of the initial mRNA molecule sequence information will be obtained. So-called tag-based approaches in mRNA detection [Harbers M. and Carinci P., Nat Methods 2, 495-502 (2005)] commonly rely on sequencing defined regions within mRNA molecules to facilitate reproducible and consistent data analysis. Advanced approaches use sequence information obtained from both ends of mRNA molecules, which mark the borders of expressed regions in genomes [Ng P. et al., Nat Methods 2, 105-111 (2005), Bashir A. at al., PLoS Computional Biology 4, e1000051 (2008)]. Having sequence information from both ends of RNA transcripts provides a better view on the entire transcribed regions within genomes and essential information for identifying regulatory regions involved in the control of gene expression. In addition, sequencing tags from both ends of mRNAs proved instrumental for monitoring trans-splicing events.

Alternatively, the power of the next-generation sequencing has enabled new approaches for shotgun sequencing of mRNA pools [Cloonan N. et al., Nature Meth published online on May 30, 2008, Mortazavi A. et al., Nature Meth published online on May 30, 2008, Wilhelm B. T., et al., Nature published online on May 18, 2008]. Shotgun sequencing of mRNA pools or RNA-Seq provides data sets that are very similar to the data obtained in whole genome tiling array experiments using labeled RNA or DNA as a hybridization probe [Kapranov P. et al., Science 296, 916-919 (2002)]. Commonly both types of experiments identify expressed exons, but are limited to the respect that they can neither recognize how those exons had been assembled into full-length mRNAs nor do they reliably identify the ends of mRNA transcripts. Hence, these approaches do not provide quantitative expression data for individual transcripts.

Recent data obtained by various approaches show that much larger portions of genomes are actively expressed than originally estimated from computational annotations of, for instance, the human or mouse genomes. Moreover, we had to realize that the transcriptome of a cell comprises many more RNA species than originally known to a person skilled in the art. A “transcriptome” is defined as the “total messenger RNA expressed in a cell or tissue at a given point in time” according to IUPAC Glossary [http://sis.nlm.nih.gov/enviro/iupacglossary/glossaryt.html]. However, it seems reasonable to extend this definition to include other RNA species, many of which having new or thus far even unknown functions.

The RNA pool of a cell may contain, but is not limited to, the following RNA species:
mRNA: Messenger RNA (mRNA) are commonly viewed as coding RNA transcripts that encode the information for protein synthesis. Recently it has been found that many mRNA molecules lack any coding potential, but rather function by other means as non-coding RNA. Therefore a definition of “mRNA” should consider their synthesis by RNA polymerase II and their specific modification at the 5′ end by the so-called Cap structure. Many mRNAs are also modified at their 3′ end by addition of a polyA tail. However, it has to be noticed that may be up to 40 or even 50% of all mRNA molecules could lack any polyA tail at their 3′ end.
ncRNA: Non-coding RNA (ncRNA) is a general term for all RNA molecules that lack any coding potential as needed in protein synthesis. ncRNAs can be expressed by RNA polymerase I, RNA polymerase II or RNA polymerase III, and may be processed to yield functional RNA molecules such as for instance some of the short RNAs mentioned below.
tRNA: Transfer RNAs (tRNA) are an essential part of protein biosynthesis at ribosomes, where tRNAs provide the matching amino acid for elongation of the polypeptide chain.
rRNA: Ribosomal RNAs (rRNA) are central components of ribosomes, where they have structural and catalytic functions for protein biosynthesis. In a typical cell, rRNA contributes the vast majority of all cellular RNA.
snRNA: Small nuclear RNAs (snRNA) are small RNA molecules of 100-300 nucleotides found in the nucleus of eukaryotic cells. They play important roles in RNA splicing, regulation of transcription, and the maintenance of telomeres.
snoRNA; Small nucleolar RNAs (snoRNA) are small RNA molecules that guide the chemical modification of rRNA and other RNA molecules by methylation and pseudouridylation.
Guide RNA: Guide RNA is a general term for RNAs in RNA/protein complexes that “guide” the complexes by sequence-specific hybridization to matching target sequences.
miRNA: Micro RNA (miRNA) are small RNA molecules of some 21-23 nucleotides that have in part reverse complementary sequences to mRNAs. They can block translation of their target RNAs or facilitates mRNA cleavage.
siRNA: Small interfering RNAs (siRNA) are double-stranded RNA (dsRNA) molecules of 20 to 25 bp that are derived from cleaved dsRNA viruses or small hairpin RNAs (shRNA).
piRNA: PIWI-interacting RNA (piRNA) are composed of 26 to 31 nucleotides and are found in ovaries and testes. They are structurally distinct from miRNAs and siRNAs by 2′-O-methylation at their last or very 3′-end nucleotides.
tasiRNA: Trans-acting small interfering RNAs (tasiRNAs) of 21 nucleotides have been found in plants.
tmRNA: Transfer-messenger RNAs (tmRNA) combine features of tRNAs and mRNAs and are ubiquitously found in all bacteria, in certain phage, mitochondrial and plastidial genomes.
Long ncRNAs: Macro-ncRNA or macroRNA of up to 100 kbp have been identified by computational analysis of cDNAs.

Although tag-based and shotgun approaches in mRNA profiling and methods for short RNA detection [Einat P., Methods Mol Biol. 342, 139-57 (2006)] are gaining increasing attention, none of the present procedures matches the requirements for whole transcriptome analysis. Limitations in the present art are restricting, for example:
Analysis of all RNA molecules within a cell or a biological sample: Recent evidence points at direct interactions between different RNA molecules within cells that can directly facilitate regulatory processes. Presently different experiments have to be conducted focusing at Individual RNA species, making it difficult or even impossible to understand co-expression/coexistence and relative expression ratios between interacting RNA molecules.
The need for mRNA or cDNA fragmentation during template preparation may eliminate important information no longer available for sequence analysis. This is particular true for the use of random priming to drive cDNA synthesis, which leads to a loss or underrepresentation of 3′ ends in libraries.
Complicated procedures prior to sequencing lead to a bias and uneven representation of RNA molecules in samples. Here in particular long transcripts as commonly found for mRNAs are underrepresented in most cDNA libraries.
Extended manipulation steps prior to sequencing require large amounts of starting martial or depend on excessive amplification of the template prior to sequencing. Again, amplification steps for example using the PCR method lead to an underrepresentation of long mRNA within cDNA libraries.
Present approaches lack internal controls to monitor the yields during sample preparation. A better standardization of the sample preparation prior to sequencing would provide superior means to directly compare quantitative expression values obtained by digital expression methods such as SAGE, 5′ SAGE, CAGE and alike [Harbers M. and Carninci P. Nat Methods 2, 495-502 (2005)].

The present invention addresses at least some of such limitations in the present art and provides new solutions to RNA processing for direct sequencing on a solid support. The present invention, for the first time, provides methods for monitoring all RNA species within a sample and sequencing so-called “Universal Libraries” for the analysis of entire transcriptome. Moreover, the ability to directly labeling RNA species gives access to internal standards for monitoring the yields during the entire sample preparation process. Hence, the present invention greatly extends the use of the next-generation sequencing technology in expression profiling, and will make essential contributions to life science and medical research. In particular, the ability to analyze the RNA content of individual cells by isolating the RNA content from a single cell and forward it to direct analysis by single molecule detection will emphasize the power of the invention. Transcriptome-wide variations at a very low level can cause fluctuations in protein levels in mammalian cells. Such “non-genetic cell individualities” have been linked to lineage choice in progenitor cells [Chang H H et al., Nature 453, 544-547 (2008)], and as such could be of key importance to understand situations of medial importance. Hence it is foreseen that the invention will enable direct analysis of single cells and monitoring new RNA species as particularly at high demand in tumor prognosis and diagnosis of other diseases.

### SUMMARY OF THE INVENTION

The present invention provides a method for introducing functional groups at the 3′ end of RNA molecules to facilitate direct binding to a solid support, so as to make it possible to conduct further manipulations of the RNA molecules on such solid support. Manipulation of molecules on a solid support greatly reduces loss of materials in successive manipulation and purification steps. The present invention enables analysis of very small amounts of RNA. In a preferable embodiment, the analysis is possible on a pool of RNA obtained from a single cell.

The present invention also provides a method for chemical modification of diol groups to introduce labels to RNA molecules having one or more open diol groups. This reaction may occur in solution or may be conducted on a solid support. Different labels can be used to practice the invention, where the labeling group has features that allow for its direct detecting. Preferably, the label is a fluorescence group or fluorophore. Preferably, detection of the label does not interfere with other labels used in a sequencing reaction.

The present invention provides a method for introducing labels to groups specific for the 3′ end of RNA molecules. Manipulations of labeled molecules occur on a solid support, and the labels introduced prior to binding to the solid support or while being present on the solid support are used to locate modified RNA molecules on the surface, to monitor the integrity of specific RNA species within the sample, and to directly analyze data.

The invention provides a method for removing the very or last 3′ nucleotides or nucleotides at the very or last 3′ end from RNA molecules to prepare RNA molecules having free 3′ ends for modification.

Moreover, having a method for direct labeling of RNA molecules enables the preparation of internal standards to monitor yields for sample preparation and improves sequencing efficiency. Such internal standards may be of different length or different nucleotide composition to monitor distinct RNA species and characteristics of the process. RNA molecules labeled by means of the invention can be prepared in a separate reaction, quantified, stored, and added to biological samples as needed to conduct an experiment. Hence, RNA molecules labeled by means of the present invention can be sold as commercial products in their own right or as part of reagent kits.

The present invention provides, in particular, a method for specifically labeling full-length mRNA within a pool of RNAs. Those full-length mRNAs are marked by the presence of a Cap structure at their 5′ end. The Cap structure allows for introducing a second label at the 5′ end of full-length mRNAs that cannot be found at any other RNA species. Hence the invention provides a method for introducing two labels to full-length mRNA molecules as compared to one label for other RNA species within the RNA pool. In another embodiment, the invention provides a method for selectively labeling only full-length mRNA molecules within a pool of RNA molecules, whereas all other RNA species within the RNA pool do not carry any label. The ability to labeling full-length mRNAs on a surface is essential to recognize sequences corresponding to the S′ ends of mRNA. In addition, scattered full-length mRNA molecules on a surface provide patterns to distinguish between different solid supports. Hence, pattern recognition enables reproducible Identification of individual solid supports that can be used in re-sequencing or extended sequencing experiments.

The present invention provides a method for obtaining sequence information from both ends of RNA molecules. In a first sequencing reaction, a reverse transcription reaction reveals sequence information from the 3′ end of RNA. Sequencing of RNA by reverse transcription yields short cDNA molecules having sequence complementary to the 3′ end of RNA. These cDNAs are extended in a second reverse transcription reaction to reach the 5′ end of the RNA templates. Hence, the invention provides cDNA molecules having complementary sequence to RNA templates and attached to the solid support. The cDNA molecules obtained in the reverse transcription reaction can be separated from the RNA template. Preferably, cDNA molecules remain attached to the solid support, whereas the RNA templates are washed away. The 3′ ends of cDNAs (corresponding to the 5′ end of RNA) are modified to introduce a new priming site. This priming site is used to drive a second sequencing reaction to obtain sequence information from the S′ end of RNA. Hence two sequencing reactions are possible, where one sequencing reaction makes use of RNA sequencing and the other sequencing reaction makes use of DNA sequencing.

In a different embodiment, the present invention provides a method for obtaining sequence information only from the 3′ end of RNA molecules within an RNA pool. According to this embodiment, the RNA sequencing step is carried out after the RNA template has been bound to the solid support. After obtaining sequence information from the 3′ end, partial cDNAs may be extended on the solid support to prepare a full-length cDNA in a reverse transcription reaction. Single-stranded cDNA may be modified to introduce a priming site at the 3′ end of the single-stranded cDNA. This priming site may be used to prime the synthesis of a second cDNA strand. The second cDNA strand may be extended to the 5′ end of the cDNA on the solid support. The full-length cDNA may be released from the solid support in a chemical reaction or by means of digestion with an endonuclease. Otherwise, full-length cDNAs on the solid support may be used in another analysis including, but not limited to, performing a shotgun sequencing experiment.

In another embodiment, the present invention provides a method for obtaining sequence information only from the 5′ end of RNA molecules within an RNA pool. According to this embodiment, the RNA sequencing step is avoided, and a cDNA template is synthesized on the solid support having sequence complementary to the RNA molecule. After removal of the RNA, the immobilized cDNA template is sequenced from the 3′ end corresponding to the 5′ end of RNA. While all RNA molecules can be sequenced from the 5′ end by means of the present invention, only full-length mRNA molecules are specifically labeled at the Cap structure to indicate, which of those sequences are derived from true 5′ ends of mRNA.

Moreover, the invention provides a method for obtaining extended sequence information from 5′ ends of RNA. In this mode, the invention uses adapters ligated to the 3′ end of the cDNA template on the solid support that have a recognition site for a restriction endonuclease cleaving outside of its binding site. The length of the double-stranded cDNA prepared during the sequencing cycles will limit internal digestion of the template DNA. If the recognition site is adjacent to the 3′ end of the cDNA, the matching enzyme cleaves short stretches of DNA from the 3′ end of the cDNA after synthesis of a second DNA strand in a sequencing reaction. In a repetitive cycle comprising steps for adaptor ligation to the open 3′ end of cDNA, sequencing by extending the adaptor by means of a DNA polymerase, cleaving of the 3′ end of the cDNA by means of a restriction endonuclease cleaving outside of its binding site, extended sequence information from the 3′ end of DNA, corresponding to the 5′ end of RNA, can be obtained.

After obtaining sequence information from the S′ end of RNA, partial cDNAs may be extended on the solid support in a primer extension reaction using a DNA polymerase. This primer extension reaction is primed by the second cDNA strand prepared during the DNA sequencing step, and may be used to prime the synthesis of a second cDNA strand. The second cDNA strand may be extended to the S′ end of the cDNA on the solid support. The full-length cDNA may be released from the solid support in a chemical reaction or by means of digestion with an endonuclease. Otherwise, full-length cDNAs on the solid support may be used in another analysis including, but not limited to, performing a shotgun sequencing experiment.

The present invention provides a method for analyzing sequencing data. Since full-length mRNA molecules are labeled differently as compared to all other RNA species within the RNA pool, the corresponding differences in the readout of the labels for each molecule bound to the solid support can direct data analysis. Hence, the invention provides means to specify sequence information obtained from the true S′ ends of mRNA. Thus, the present invention makes it possible to identify promoters driving RNA polymerase II-mediated transcription for a consecutive data analysis.

Moreover, the present invention offers a method for identifying and analyzing at the same time all RNA molecules within a pool of RNA molecules as obtained from a biological sample or an artificial pool of RNA molecules. Since all RNA molecules within the pool are processed in parallel, the invention enables the unbiased analysis of entire transcriptomes. Hence the invention provides a new method for describing relationships between RNA molecules within a pool of RNA molecules such as direct hybridization of regions having in part or entirely complementary sequence. In a preferable embodiment, sequences obtained by means of the invention allow for genome-wide expression analysis. In an even more preferable embodiment, sequences obtained by means of the invention are mapped to genomic sequences, where genomic sequences and their annotations guide the analysis of sequences obtained by means of the invention.

The present invention provides a method for analyzing transcriptome to describe a biological sample. Hence the invention can be applied to research and diagnostics of human, animals, plant, and microorganisms.

The invention provides means to prepare reagents for practicing the entire invention or any individual step thereof. Hence the invention provides means to produce individual reagents and reagent kits.

A first aspect of the present invention is a method for simultaneous identification and analysis of RNA molecules in a sample, comprising: preparing a solid support which has capturing oligonucleotide molecules attached onto its surface, introducing a functional group at the 3′ end of RNA molecules present in the sample, binding the RNA molecules by hybridization to capturing oligonucleotide molecules which have a sequence complementary to the sequences of the functional group and which are fixed on a solid support, and subjecting the RNA molecules to analysis as attached to the solid support.

A second aspect of the present invention is a method for simultaneous identification and analysis of RNA molecules in a sample, comprising: preparing a solid support which has capturing oligonucleotide molecules attached onto its surface, introducing a functional group at the 3′ end of RNA molecules present in the sample, labeling one or more diol groups present in the RNA molecules with a labeling molecule, binding the RNA molecules by hybridization to the capturing oligonucleotide molecules which have a sequence complementary to the sequences of the functional group, detecting the labeling molecule introduced to the RNA molecules to identify a feature, including existence or absence of a Cap structure, of each RNA molecule attached to the solid support, and subjecting the RNA molecules to analysis as attached to the solid support.

A third aspect of the present invention is a method for simultaneous identification and analysis of mRNA molecules in a sample, comprising: preparing a solid support which has capturing oligonucleotide molecules attached onto its surface, labeling one or more diol groups present in the mRNA molecules with a labeling molecule, binding the mRNA molecules to the capturing oligonucleotide molecules which have a sequence complementary to a sequence of the 3′ end of the RNA molecules, detecting the labeling molecules attached to the mRNA molecules to identify a feature, including existence or absence of a Cap structure, of each mRNA molecule attached to the solid support, and subjecting the mRNA molecules to analysis as attached to the solid support.

A fourth aspect of the present invention is a method for simultaneous identification and analysis of mRNA molecules in a sample, comprising: preparing a solid support which has capturing oligonucleotide molecules attached onto its surface, labeling the mRNA molecules with a suitable labeling molecule, binding the mRNA molecules by hybridization to the capturing oligonucleotide molecules, which have a sequence complementary to the sequence of the functional group introduced at the 3′ end of the RNA molecules, detecting two labeling molecules for a single mRNA molecule so as to determine that said single RNA molecule is full length having a labeled Cap structure and a labeled polyA tail, and subjecting the full-length mRNA to analysis as attached to the solid support.

A fifth aspect of the present invention is a method for simultaneous identification and analysis of mRNA molecules in a sample, comprising: preparing a solid support which has capturing oligonucleotide molecules attached onto its surface, labeling one or more diol groups present in the mRNA molecules with a suitable labeling molecule, binding the mRNA molecules by hybridization to the capturing oligonucleotide molecules, which have a sequence complementary to the sequence of the functional group introduced at the 3′ end of the RNA molecules, priming reverse transcription of the mRNA molecules attached to the solid support to obtain cDNA strands complementary to the mRNA molecules so as to form DNA-RNA hybrids, subjecting the DNA-RNA hybrids to RNase I treatment for removal of their single stranded portion, detecting the labeling molecule to identify any cDNA strands in the hybrids that have reached the 5′ end of any full-length mRNA, washing away the RNA molecules so as to obtain single-stranded cDNA, and adding a priming site to the single-stranded cDNA molecules which represent full-length mRNA to analysis for sequencing of the 3′ end of such cDNA while it remains attached to the solid support.

### BRIEF DESCRIPTION OF THE DRAWINGS

FIG. 1: Removal of blocked 3′ ends from RNA

FIG. 1 provides a schematic drawing on the steps involved in removing the last or very 3′-end nucleotides from an RNA molecule. In a first step, a double-stranded DNA adapter having an overhanging region of random sequence (NNNNN) is hybridized to the 3′ end of RNA. In a second step, RNase H digests the RNA portion within the DNA-RNA hybrid, thus removing the nucleotides at the 3′ end of the RNA molecule. The double-stranded DNA adapter may be ligated to the 3′ end of RNA, if such 3′ end is not blocked.

FIG. 2: RNA pool of a cell or biological sample

FIG. 2 provides schematic drawings of RNA species within an RNA pool obtained from a cell or biological sample: 1) Full-length mRNA having a Cap structure at the 5′ end and polyA tail at the 3′ end. 2) Truncated mRNA without Cap structure, but having a polyA tall at the 3′ end. 3) A potentially full-length mRNA molecule having a Cap structure but lacking a polyA tail. This molecule may represent non-polyadenylated mRNA or mRNA truncated at the 3′ end, 4) An RNA molecule having no modifications at the 5′ and 3′ end. This includes truncated RNA molecules as well as short RNA molecules derived from a maturation process. 5) An RNA molecule modified in the 2′ position of the last or true 3′-end nucleotide (marked by a hatched circle), such as piRNA. 6) An RNA molecule having a blocked 5′ end (marked by a hatched circle), such as RNA polymerase I derived transcript having a triphosphate group at the 5′ end. The 5′ end modification is not a Cap structure.

FIG. 3: 3′ end modification of RNA within RNA pool by polyadenylation

FIG. 3 provides schematic drawings of the RNA species described in FIG. 2 after a polyA tail (underlined in the figure) has been added to the open 3′ end of such RNA species. The addition of a polyA tail made from ribonucleotides will introduce an open diol group at the 3′ end of the polyA tail.

FIG. 4. 3′ end modification of RNA within RNA pool by oligonucleotide ligation

FIG. 4 provides schematic drawings of the RNA species described in FIG. 2 after an oligonucleotide group has been ligated to the open 3′ end of such RNA species. Such oligonucleotide group may have two regions of different sequence or function as indicated by “X” and “Z” in the figure “Z” may represent an “Identifier Sequence” to mark individual RNAs or pools of RNAs within the same sample. Sequences within “Z” could further function as a functional group to facilitate binding to a solid support. “X” may have a sequence that is not complementary to sequences presented on the solid support for attaching the RNA to such solid support. Hence, nucleotides within “X” will not hybridize while binding RNA to solid support and as such will remain as single-stranded RNA at all times. Moreover, “X” and “Z” can indicate the RNA and DNA portion in an RNA-DNA hybrid oligonucleotide. Preferably, “Z” is made of ribonucleotide, and “X” is made of desoxyribonucleotide. If the nucleotide at the very or true 3′ end is a desoxyribonucleotide, the modified RNA molecule has no diol group at the 3′ end.

FIG. 5: 3′ end modification of RNA within RNA pool by ligation of modified oligonucleotide

FIG. 5 provides schematic drawings of the RNA species described in FIG. 2 after modified oligonucleotide has been ligated to the open 3′ end of such RNA species. Such modification (marked by solid circles in the figure) may be a functional group for binding the RNA to a solid support, may function as a label to mark the origin of individual RNAs or RNA pools within the same sample, or may be used to mark the location of the RNA molecule on the solid support.

FIG. 6: Labeling of open diol groups within RNA molecules

FIG. 6 provides schematic drawings of the RNA species described in FIG. 3 having a polyA tail at their 3′ end after all diol groups within such RNA molecules have been chemically modified to introduce a label (marked by an open star in the figure).

FIG. 7: Labeling of diol groups within RNA molecules

FIG. 7 provides schematic drawings of the RNA species described in FIG. 4 having an oligonucleotide at their 3′ end after all diol groups within such RNA molecules have been chemically modified to introduce a label (marked by an open star in the figure).

FIG. 8: Labeling of diol groups within RNA molecules (blocked 3′ ends)

FIG. 8 provides schematic drawings of the RNA species described in FIG. 5 having a modified oligonucleotide at their 3′ end after all diol groups within such RNA molecules have been chemically modified to introduce a label (marked by an open star in the figure). Here it is assumed that addition of a functional group at the 3′ end has destroyed the diol group at the 3′ end of RNA.

FIG. 9: Use of two different labels within RNA molecules with blocked 3′ ends

FIG. 9 provides schematic drawings of the RNA species described in FIG. 8 having a label attached to the oligonucleotide at their 3′ end. As indicated by the triangle, this label may be different from the label attached to the diol groups within RNA (marked by an open star in the figure).

FIG. 10: RNA binding to solid support and fill-in reaction

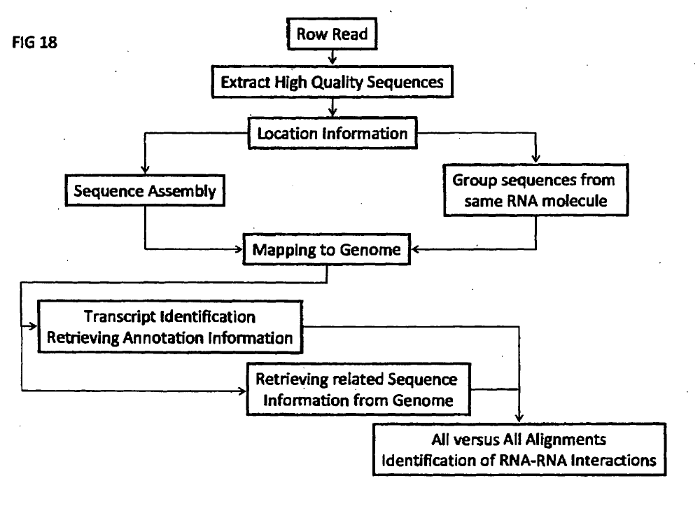

In FIG. 10, molecule 1 is a schematic drawing of a full-length mRNA molecule, and molecule 2 is a schematic drawing of an RNA molecule lacking a Cap structure at the 5′ end. Such RNA molecules may bind to a solid support by hybridization to capturing oligonucleotides having a complementary sequence to sequences at the 3′ end of RNA. Such sequences include, but are not limited to, polyA tails at the 3′ end of RNA hybridizing to oligo-dT sequences on the solid support. After the hybridization reaction, the oligo-dT oligonucleotide can prime an extension reaction using a reverse transcriptase. Providing only dTTP in the reaction mixture, the reaction will terminate at the first nucleotide within the RNA that is not an “A”. Hence, the fill-in reaction will terminate at the 3′ end of the RNA. Similarly, any other homopolymer made of polyC, polyG, or polyT may be added to the 3′ end of RNA and used in the hybridization and fill-in reactions. When modifying the 3′ end of RNA by ligation of an oligonucleotide, such oligonucleotide can hybridize to the capturing oligonucleotides on the solid support having complementary sequence. The oligonucleotides can be designed in such a way that a fill-in reaction to reach the 3′ end of RNA may not be required, if the target RNA molecule has no polyA tail.

FIG. 11: First sequencing of RNA template and extension reaction

FIG. 11 provides schematic drawings of the reaction products derived from the reactions described in FIG. 10, Oligo-dT primers have been extended in the previous reaction to reach the 3′ end of RNA molecules. Such extended oligo-dT primers can be used to prime a sequencing reaction to obtain sequence information from the 3′ end of RNA molecules. The sequencing reaction may be terminated after a number of reaction cycles, where the number of reaction cycles is smaller than the number of nucleotides within the RNA molecule. After completion of the sequence reactions, a cDNA having complementary sequence to the RNA molecule can be synthesized in a fill-in reaction using a reverse transcriptase. Preferably, the cDNA will be extended to the 5′ end of the RNA, and as such will be a full-length cDNA, Optionally, the sequencing step may be omitted, and oligo-dT primers are extended in a fill-in reaction to reach the 5′ ends of the RNA molecules.

FIG. 12: RNA removal and linker ligation/G-tailing reaction

FIG. 12 provides schematic drawings of the reaction products derived from the reactions described in FIG. 11. cDNA molecules synthesized by extension of the oligo-dT primer will remain attached to the solid support after the removal of the RNA template. After removal of the RNA template, the cDNA molecules on the solid support may be modified at their 3′ end to introduce a priming site. The priming site can be made of an oligonucleotide ligated to the 3′ end or can be made of a homopolymer added in an extension reaction. The oligonucleotide and the homopolymer may carry a label to mark the position of modified cDNA molecules on the solid support.

FIG. 13: Second sequencing of DNA template

FIG. 13 provides schematic drawings of the reaction products derived from the reactions described in FIG. 12. The priming site introduced at the 3′ end of the cDNA is used to drive a sequencing reaction to obtain sequence information from the 5′ end of the original RNA template or to prepare a second cDNA strand having a sequence complementary to the cDNA bound to the solid support.

FIG. 14: Dual labeling sequencing

FIG. 14 provides schematic drawings of the different reaction steps required to obtain sequence information corresponding to the 5′ end of RNA by dual labeling sequencing: 1) A primer is immobilized on a solid support. 2) An RNA molecule is hybridized to the primer (capturing oligonucleotide) attached to the solid support. The RNA molecule as shown in the drawing is a full-length mRNA carrying a label at the 5′ and 3′ end. These labels were introduced in a chemical reaction modifying diol groups within the Cap structure and at the 3′ end of RNA. 3) Detection of labeling groups for molecules bound to the surface, which indicates the location of RNA molecules bound to primer sites. 4) A primer extension reaction is performed by a reverse transcriptase to synthesis a full-length cDNA having complementary sequence to the RNA template. 5) DNA-RNA hybrids on the solid support are treated by RNase 1. RNase I will remove all regions made of single-stranded RNA. As shown in the figure, this treatment will remove the label at the 3′ end of RNA. 6) Detection of labeled groups for molecules bound to the surface. After removal of the label at the 3′ end of RNA, this detection step will only recognize DNA-RNA hybrids where the cDNA has reached the 5′ end of full-length mRNA. 7) The RNA strand is removed from the DNA-RNA hybrids. 8) A priming site is added to the 3′ end of the cDNA. 9) A sequencing primer is hybridized to the priming site at the 3′ end of the cDNA. 10) The sequencing primer hybridized to the priming site at the 3′ end of the cDNA drives a sequencing reaction to obtain sequence information from the 5′ end of the original RNA molecule.

FIG. 15: Class IIs enzyme mediated primer walking

FIG. 15 provides schematic drawings for the reaction steps required to obtain sequence information from the 5, end of RNA or DNA. 1) A priming site (marked as a hatched box in the figure) is added to the 3′ end of a DNA molecule attached to a solid support. The priming site has adjacent to the 3′ end of the DNA molecule a recognition site (marked as a white box in the figure) for a restriction endonuclease that cuts at a position outside of restriction endonuclease's binding site including, but not limited to, a class IIs restriction enzyme, 2) A sequencing primer hybridizes to the primer site and drives a sequencing reaction. 3) The restriction endonuclease that cuts at a position outside of its binding site is used to remove a DNA fragment from the end of the DNA molecule attached to a solid support. 4) A priming site is added to the open end of a DNA molecule attached to a solid support. The priming site has adjacent to the 3′ end of the DNA molecule a recognition site for a restriction endonuclease that cuts outside of its binding site. 5) A sequencing primer hybridizes to the primer site and drives a new sequencing reaction. Reaction steps 1 to 5 can be repeated in a cycling process to obtain extended sequence information from a DNA molecule attached to a solid support. Since the restriction endonuclease that cuts outside of its binding site can only cut double-stranded DNA, the enzyme cuts off only DNA regions for which the second cDNA strand had been synthesized during the sequencing reaction, and not within single-stranded cDNA.

FIG. 16: Chemical structure of compounds used in diol labeling reaction

FIG. 16 provides an example for a structure of a representative chemical compound for use in a diol labeling reaction. The chemical structure is composed of a “Fluorophore”, a “Linker”, and a “Hydrazide” group.

FIG. 17: Diol labeling reaction

FIG. 17 provides a schematic representation of the reaction steps to modify diol groups in a labeling reaction. Diol groups (2′, 3′-terminal vicinal dihydroxy groups) as indicated in the figure can be found within the Cap structure (7-Methylguanylate) of full-length mRNA and at the open 3′ end of RNA molecules. This includes RNA molecules extended by adding ribonucleotides to the 3′ end. In a first reaction step, the diol groups are oxidized by sodium periodate (NaIO₄) prior to reacting with the hydrazine group within the Fluorophore Hydrazine in a second reaction step.

FIG. 18: Workflow of bioinformatics analysis

FIG. 18 outlines key steps for the computational analysis of sequences obtained by means of the invention: High quality sequences are extracted from row sequencing reads; high quality reads are linked to the location information obtained by the readout of the labels; sequences obtained from the same location are grouped, and where possible assembled into longer sequences; sequences within each group or from assembled sequences are mapped to a suitable genome; mapping positions in the genome identifies the transcripts and provides annotation information on the transcribed regions; sequence information from the transcribed regions can be retrieved from genome sequences; and sequence information from transcribed regions is used in alignments to identify interactions between RNA molecules having complementary or partially complementary sequence.

### DETAILED DESCRIPTION OF THE INVENTION

In the following section, the present invention will be described in detail. All terms and abbreviations shall have a standard meaning known to a person skilled in the art unless otherwise defined, and all references cited here shall be incorporated herein by reference. This includes the content of the internet pages cited herein as accessed as of June 2008.

The terms “purity”, “enriched”, “purification”, “enrichment”, or “selection” are used interchangeably herein and do not require absolute purity or enrichment of a product but rather are intended as relative definitions. The terms “specific”, “preferable”, or “preferential” are used interchangeably herein and do not require absolute specificity of a DNA or RNA hybridization probe, or an enzyme for its substrate or an activity, but rather they are intended to have relative definitions which include the possibility that an enzyme may have low or lower affinity to other compounds related or unrelated to its substrate.

Similarly, the terms used to name an enzymes an enzymatic activity, and a compound are used herein to describe the function or activity of such a component, but do not require the absolute purity of such enzymes or components. Thus, any mixture containing such an enzyme, enzymatic activity, a compound or mixtures thereof with other components of the same, related or unrelated function are within the scope of the invention. Similarly, DNA or RNA molecules may function in a specific manner as functional group, hybridization probes, primers, or capturing oligonucleotides, and as such are related to as “complementary sequences” for the purpose of the invention, or in experiments where such probes, primers, or capturing oligonucleotides are applied for the detection or binding to related nucleic acid molecules, even where such a probe and the target molecule may be distinct by naturally occurring or artificially introduced mutations in individual positions.

For manipulation, detection, or analysis including performing a sequencing reaction, nucleic acid molecules may be attached or otherwise bound to a solid support. Materials for the use as a solid support can include any solid material with which components can be associated directly or indirectly. Such materials include but are not limited to acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, or any combination thereof. Solid supports further include thin film, membrane, bottles, dishes, fibers, woven fibers, shaped polymers such as tubes, particles, beads, microparticles, or any combination thereof. A solid support may have different shapes, preferable the shape is a slide or a bead. A solid support may further have holes or depressions to perform reactions at defined locations in an arrayed format on or within the solid support. Reactions on a solid support may be carried out in the presence of one or more additives. Such additives may help to keep beads in suspension or otherwise increase the fidelity of enzymes acting in close proximity to the solid support. The additive may be a chemical compound, a polymer, a polysaccharide, a protein, a chaperon, or any mixture thereof.

The expressions “DNA”, “RNA”, “nucleic acid”, and “sequence” encompass nucleic acid materials themselves and are not restricted to particular sequence information, vector, phage, phagemid, BAC, YAC, or any other specific nucleic acid molecule. The term “nucleic acid” is also used heroin to encompass naturally occurring nucleic acids, artificially synthesized or prepared nucleic acids, and any modified nucleic acids into which at least one or more modifications have been introduced by naturally occurring events or through approaches known to a person skilled in the art. Similarly, a “tag” and an “Identifier Sequence” according to the invention can be any region of a nucleic acid molecules as prepared by means of the invention, where the terms “tag” and/or an “Identifier Sequence” as used herein encompasses any nucleic acids fragment, no matter whether they are derived from naturally occurring, artificially synthesized or prepared nucleic acids, any modified nucleic acids into which at least one or more modifications have been introduced by naturally occurring events or through approaches known to a person skilled in the art. Furthermore, the terms “tag” and/or an “Identifier Sequence” do not relate to any particular sequence information or their composition but to the nucleic acid molecules as such. A tag and an Identifier Sequence carry features for detection or modification. A tag and an Identifier Sequence may be made of nucleic acid or chemical compound. The chemical compound may be a dye, or more preferably a fluorescent dye. A fluorescent dye can be any dye including, but not limited to, dyes excited with visible light or excited with UV light. The invention is not limited to the use of any particular dye, but it is within the scope of the invention to select different dyes as commercially available or otherwise having preferable features for use to practice the invention. Preferably, sets of dyes are selected that allow for a simultaneous detection of more than one dye in the same reaction. A set of dyes that can be detected at the same time includes but is not limited to Cy3, Cy5, FAM, JOE, TAMRA, ROX, dR110, dR6G, DTAMRA, dROX, or any mixture thereof. Any of those dyes may be used individually or in any combination to practice the invention. More preferably, a dye should allow for single molecule detection. Examples for the use of fluorescence methods in single molecule detection have been described by Joo C et al., Annu Rev. Biochem. 77, 51-76 (2008).

The invention encompasses handling single-stranded as well as double-stranded nucleic acid molecules in the form of linear nucleic acid molecules, Double-stranded DNA means any nucleic acid molecules each of which is composed of two polymers formed by deoxyribonucleotides and in which the two polymers have substantially complementary sequences to each other allowing for their association to form a dimeric molecule. The two polymers are bound to one another by specific hydrogen bonds formed between matching base pairs within the deoxyribonucleotides. Similarly, a DNA molecule can form a double-stranded hybrid molecule by hybridizing to an RNA molecule having complementary sequence. Any DNA molecule composed only of one polymer chain formed by two or more deoxyribonucleotides having no matching complementary DNA molecule to associate with is considered to be a single-stranded DNA molecule for the purpose of the present invention, even if such a molecule may form secondary structures comprising double-stranded DNA portions.

As used interchangeably herein, the terms “nucleic acid molecule(s)”, “polynucleotide(s)” and “oligonucleotide(s)” include RNA and DNA regardless of whether they are single or double-stranded, coding or non-coding, complementary or not, sense or antisense, or regardless of whether or not they include ribonucleotides and desoxyribonucleotides to form RNA-DNA hybrid molecules. In particular, it encompasses genomic DNA and complementary DNA, so-called cDNA, which are transcribed or non-transcribed, spliced or not spliced, processed, incompletely spliced or processed, independent from its origin, cloned from a biological material, or obtained by means of synthesis.

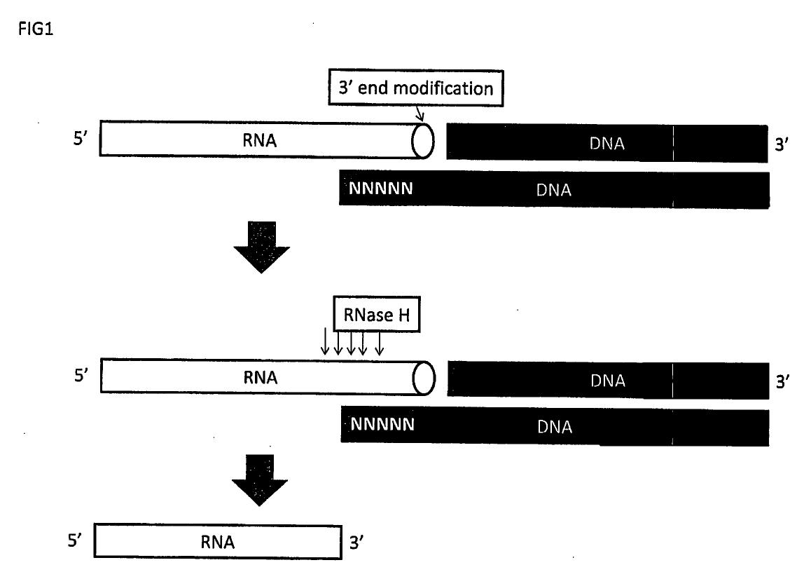

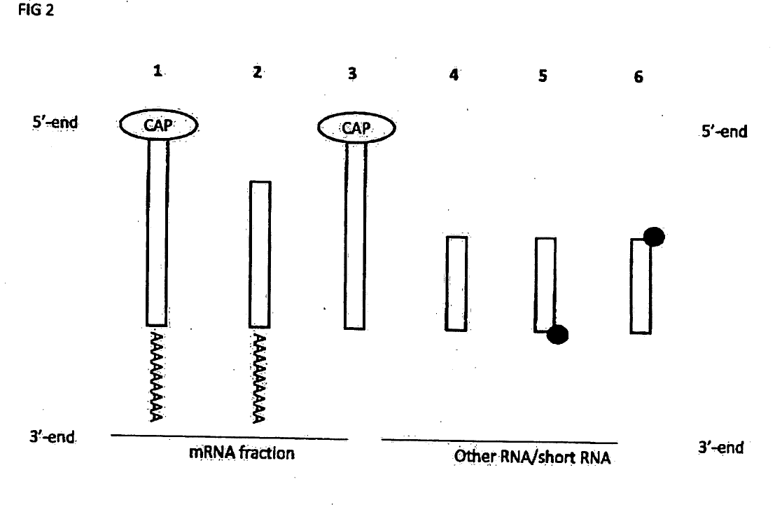

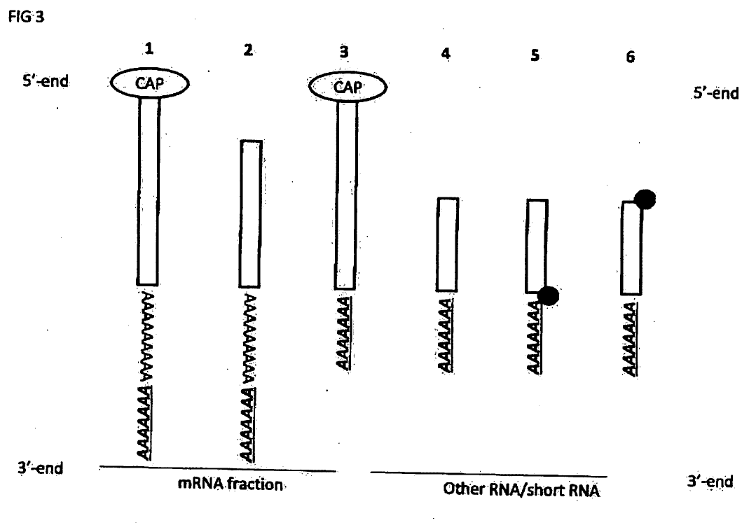

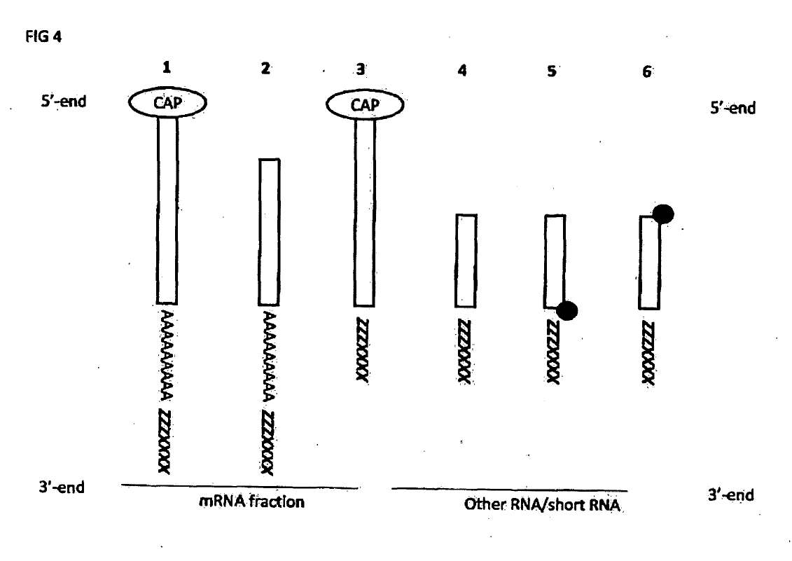

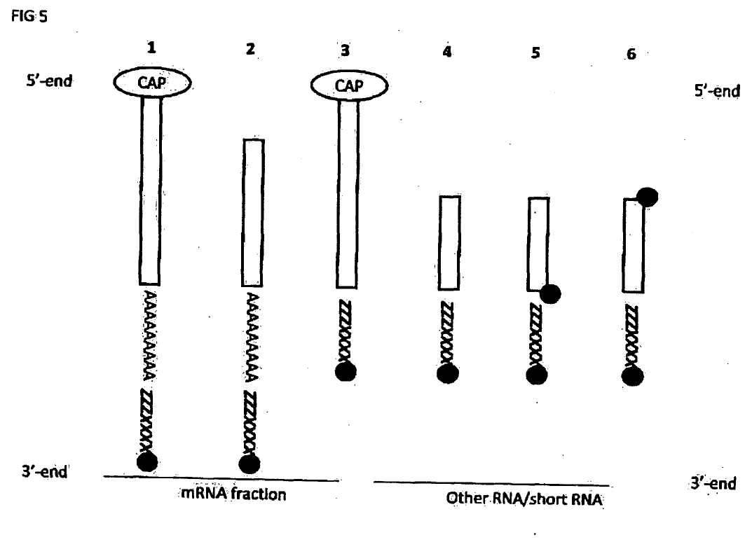

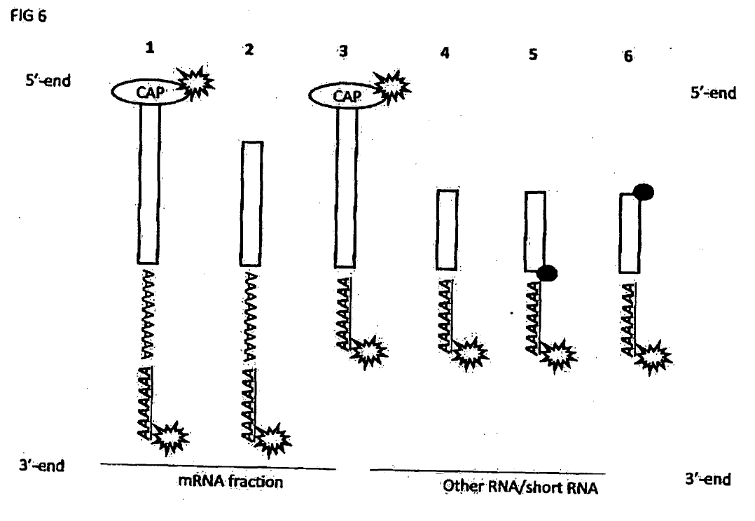

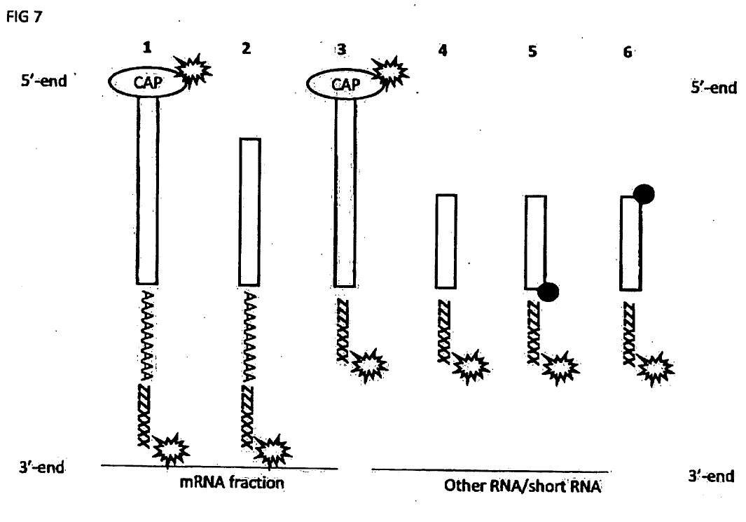

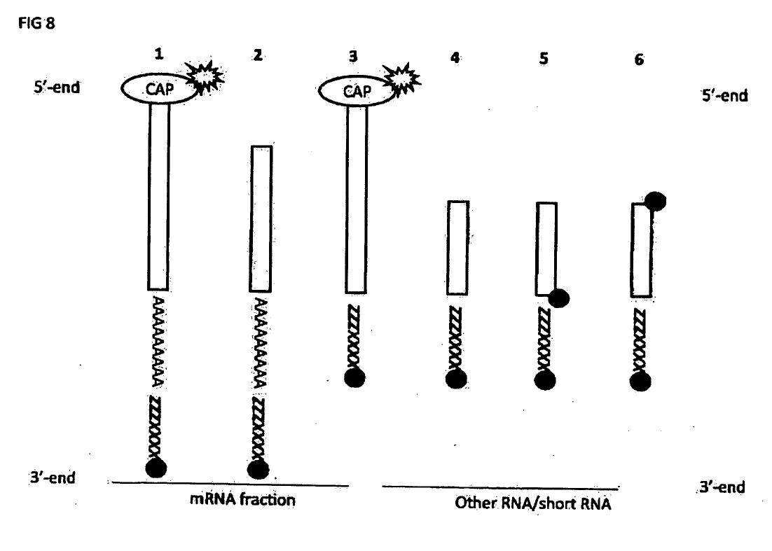

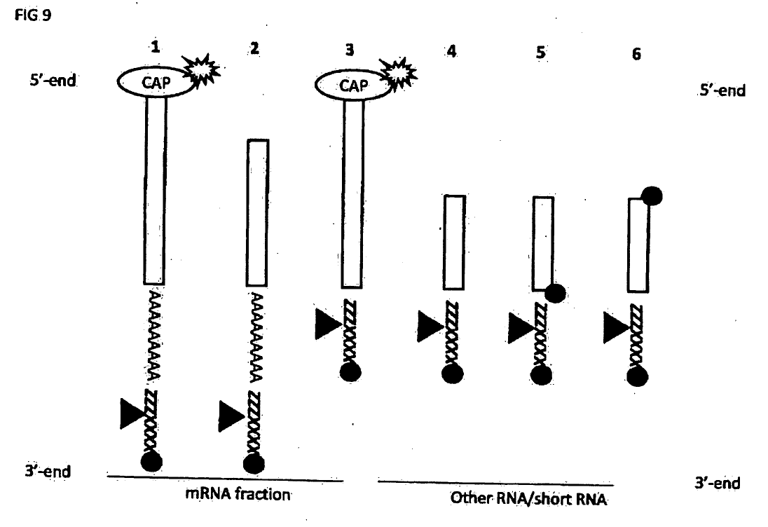

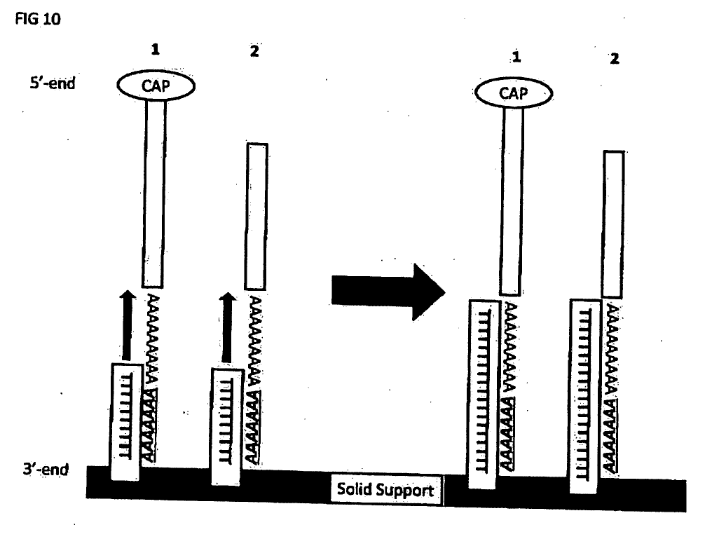

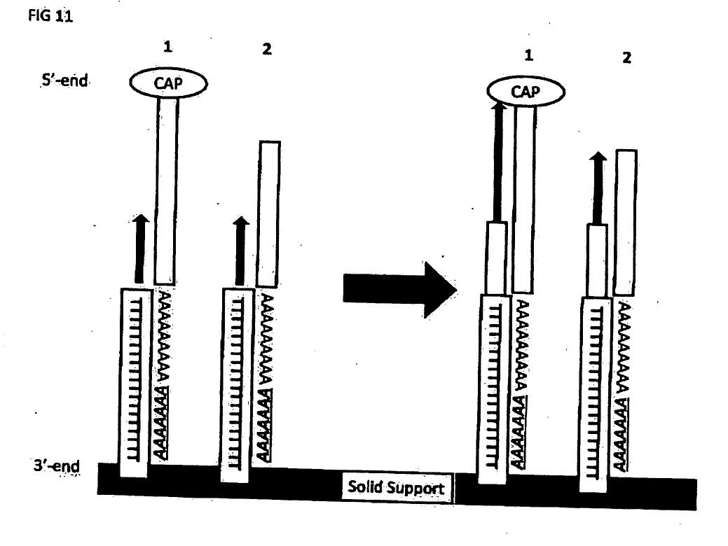

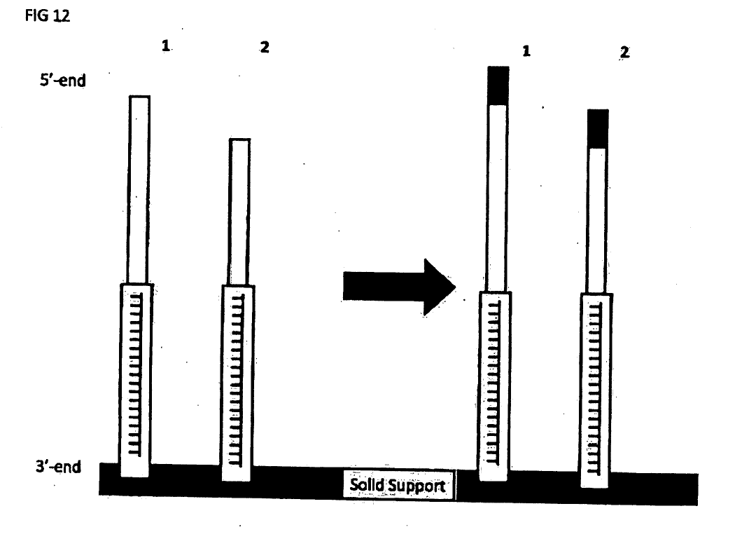

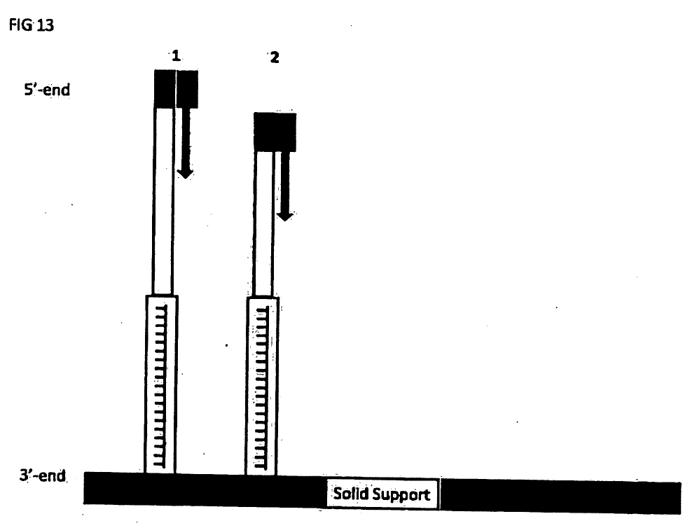

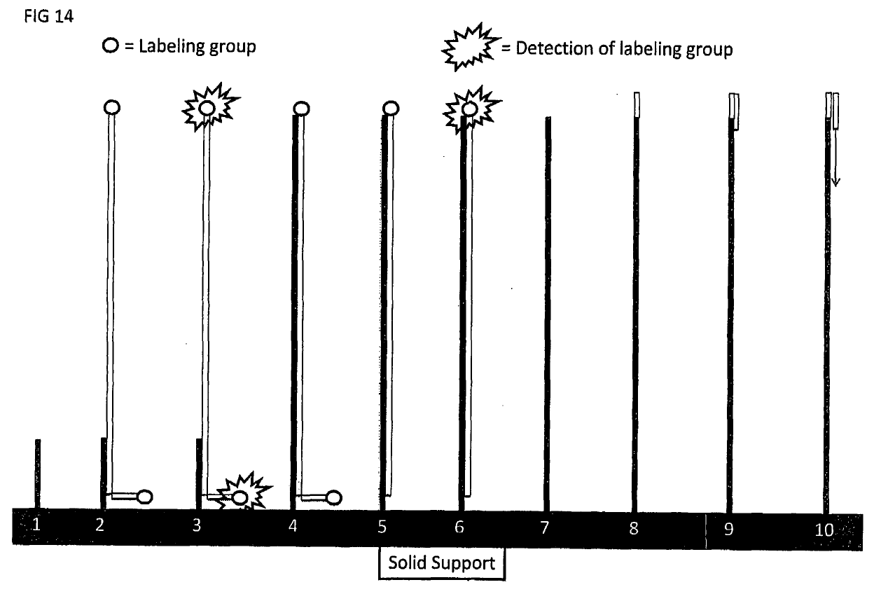

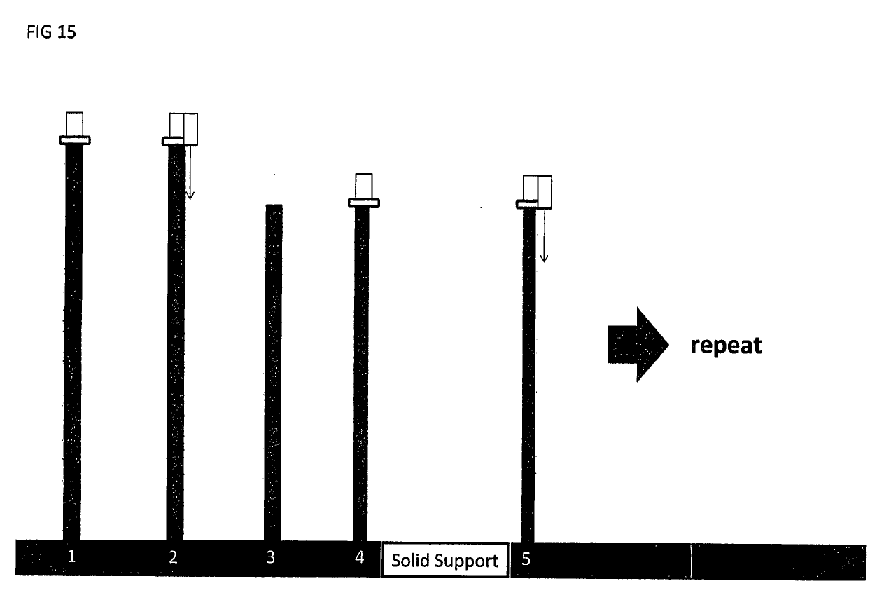

RNA, for the purpose of the present invention, is considered a single-stranded nucleic acid molecule even where such a molecule may form secondary structures comprising double-stranded RNA portions. Single-stranded RNA molecules may form hybrids together with other RNA molecules, or have in part or over their entire length complementary sequences to form in part or over their entire sequence double-stranded RNA molecules. In particular, RNA encompasses, for the purpose of the present invention, any form of nucleic acid molecule comprised of ribonucleotides, and does not related to a particular sequence or origin of the RNA. Thus RNA can be transcribed in vivo or in vitro by artificial systems, or non-transcribed, spliced or not spliced, processed or not processed, incompletely spliced or processed, independent from its natural origin or derived from artificially designed templates, mRNA, ncRNA, tRNA, rRNA, snRNA, snoRNA, GuideRNA, miRNA, siRNA, piRNA, tasiRNA, tmRNA, macroRNA, macro-ncRNA, obtained by means of synthesis, or any mixture thereof. In particular, the invention can be used to distinguish between RNA molecules having a Cap structure at their 5′ end and those RNA molecules that lack a Cap structure at their 5′ end. Some RNA molecules may have a polyA tail at their 3′ end or are otherwise modified at their 5′-, 3′-, or 2′ ends. TABLE 1 (shown below) groups RNA molecules commonly found in a sample according to their features at the 5′ and 3′ ends: 1) Full-length mRNA molecules having a Cap structure at the 5′ end and polyA tail at the 3′ end. 2) Truncated mRNA molecules without Cap structure, but having a polyA tail at the 3′ end. 3) Potentially full-length mRNA molecules having a Cap structure but lacking a polyA tail. These molecules may represent non-polyadenylated mRNA or mRNA molecules truncated at the 3′ end. 4) RNA molecules having no modifications at the 5′ and 3′ end. They include truncated RNA molecules as well as short RNA molecules derived from a maturation process. 5) RNA molecules modified in the 2′ position such as for example piRNAs. 6) RNA molecules having a blocked 5′ end such as for example RNA polymerase I derived transcripts having a triphosphate group at the 5′ end. 7) RNA molecules with blocked 3′- and 2′ ends. The invention targets at the analysis of all RNA molecules within a sample. However, certain RNA molecules may carry modifications at their 3′ end or at their 3′ and 2′ positions of the last 3′-end nucleotide that could block any further manipulation of such RNA molecules by means of the invention. Therefore, the present invention provides the removal of the last 3′-end nucleotides of an RNA molecule in an RNase H meditated digestion step (FIG. 1) to make such RNA species accessible to perform the invention.

TABLE 1
RNA Species in an RNA Pool

| No in | | |
|---|---|---|
| Figures | Description | Labeling |
| 1 | Full-length mRNA with polyA tail | Diol group at 5′ and 3′ |
| | and Cap structure | end |
| 2 | Truncated mRNA with poly A tail | Diol group at 3′ end |
| | lacking the Cap structure | |
| 3 | Non-polyadenylated full-length | Diol group at 5′ and 3′ |
| | cDNA with Cap structure | end |
| 4 | RNA with free 5′ and 3′ end | Diol group at 3′ end |
| | (RNA fragments/degradation products) | |
| 5 | RNA with modified 2′ group | No diol group |
| 6 | RNA with blocked 5′ end | Diol group at 3′ end |
| 7 | RNA with blocked 3′ and 2′ groups | No diol group |

In order to perform the invention, nucleic acid molecules can be derived from any naturally occurring genomic DNA, RNA, an existing DNA library, or any mixture thereof. They can also be of artificial origin. The invention is not limited to the use of an individual nucleic acid molecule or any plurality of nucleic acid molecules, but the invention can be performed on an individual nucleic acid molecule or any plurality of nucleic acid molecules, regardless whether such pluralities occur in nature, which are derived from a cell, a tissue, an organism, or an existing library, or which may be artificially created. Furthermore, according to the present invention any nucleic acid molecule can be processed regardless of its origin or nature. Thus it is within the scope of the present invention that the nucleic acid molecules can be full-length molecules as compared to naturally occurring nucleic acid molecules, or any fragment thereof. Even further, it can be envisioned that such fragments of nucleic acid molecules are prepared by a random process or by a targeted dissection of nucleic acid molecules by means of an enzymatic activity with a preference for a certain sequence, or fragmentation based on the structure of the nucleic acid molecule including, but not limited to, exons and introns within transcribed regions, or a chemical reaction. A pool of nucleic acids may also be fractionated by having features for selective binding to a solid support including, but not limited to, having a polyA tail at the 3′ end of a mRNA molecule, having a Cap structure at the 5′ end of a mRNA molecule, the ability of an RNA molecule to hybridize to a genomic regions, or the ability of an RNA molecule to hybridize to another RNA molecule. Hence, the invention includes the possibility to enrich certain RNA molecules such as mRNA, RNA derived from defined locations in the genome, or RNA which is capable of physically interacting with a different RNA molecule. The selection step may occur independently from the sequencing reaction including, but not limited to, the use of a microarray platform, or it may occur on the solid support used in the sequencing process. Thus the invention provides a method for selective analysis of certain RNA molecules, though the invention is not restricted to the use of any particular starting material or RNA molecules.

The term “biological sample” includes any kind of material obtained from living organisms, dead or alive, including microorganisms, animals, and plants, as well as any kind of infectious particles including viruses and prions, which depend on a host organism for their replication. As such “biological sample” includes any kind material obtained from a patient, animal, plant or infectious particle for the purpose of research, development, diagnostics or therapy. Thus, the invention is not limited to the use of any particular nucleic acid molecules or their origin, but the invention provides general means to be applied to and used for the work on and the manipulation of any given nucleic acid.

The nucleic acid molecule can be an RNA molecule. RNA may derive from biological samples or more specifically from fluids of biological origin, such as blood or serum. For instance, it may contain viral RNA or other potential parasites from the blood of an individual human; or the RNA may be obtained from purified cells, including flow-sorted cells from dissected tissue, where cells may be labeled with a selectable fluorescent antibody for cell sorting or by the transgenic expression of a marker such as the green fluorescent protein (GFP) or by using other methods known to a person trained in the art. RNA can further be obtained by recent technologies for the isolation of individual cells including, but not limited to, laser capture microdissection or cell aspiration after micro injection. Such cells can be selected based on their morphology or biological features to drive the analysis of specific questions. Moreover, cells may be fractionated to isolate RNA from defined parts of a cell including, but not limited to, different organelles Preferably, RNA may be isolated from the cell's nucleus or the cell's cytoplasm.

Any RNA molecule as applied to perform the invention can be obtained or prepared by any method known to a person skilled in the art including, but not limited to, those described by Sambrook J. and Russuell D. W., Molecular Cloning, A Laboratory Manual, Cold Spring Harbor Laboratory Press, New York, 2001. Other protocols can be found in the public domain as for example under: http://www.protocol-online.org/. In addition, many providers offer commercial solutions and reagents to isolate RNA or DNA from a sample. For example, RNA can be isolated by purification kits including, but not limited to, TRizolR from Invitrogen, QuickExtract™ FFPE RNA Extraction Kit from Epicentre, or the PicoPure™ RNA Isolation Kit from Molecular Devices for RNA isolation from a single cell. It is within the scope of the invention that RNA may be isolated from organelles or derived from a cell fractionation experiment by any such procedure. RNA purified by such means may be further fractionated according to size or any other features suitable for enrichment including, but not limited to, a hybridization reaction.

Preferably, according to the invention, the analysis of all RNA species present within a sample is envisaged. RNA purification may be done by a method that allows the extraction of all RNA molecules from a cell, a biological sample, or an artificially prepared sample. Purified pools of RNA molecules do not have to be further fractionated to separate, for example, specific RNA molecules as often done for polyadenylated mRNAs. Moreover, the invention does not require any size fractionation of the RNA pool as commonly done for the analysis of short RNA. In specific embodiments of the present invention, however, it may be advantageous to remove certain RNA species that are not of interest to the analysis of a sample. For example, it may be desirable to remove rRNA from a sample prior to performing the present invention, as rRNA can make up for some 80% of all RNA within a cell, Ribosomal RNA from human and mouse samples can be effectively removed by the Ribominus™ kit from Invitrogen. This kit uses hybridization probes bound to a matrix having complementary sequences to rRNA. This concept can be extended to prepare reagents containing beads presenting oligonucleotides complementary to specific RNA molecules. Alternatively, enzymatic digestions using DNA fragments or oligonucleotides and RNase H can selectively remove RNA molecules [U.S. Pat. No. 6,544,741]. In combination with database searches, computational prediction of RNA structures, open reading frames, or any other features, RNase H mediated digestion can remove specific RNA species using oligonucleotides hybridizing to specified RNA motifs or cleaving off priming sites from selected RNA molecules. RNase H mediated digestion of RNA can be done at different time points, such as alter RNA isolation or after modification of RNA introducing a modification at its 3′ end. Such approaches are relevant to experimental designs focusing on certain groups of RNA molecules, or may be use to normalize the ratio of different RNA molecules within a sample.

According to the present invention, the 3′ end of RNA molecules may be modified to attach RNA molecules to a solid support. Hence, modifications at the 5′ end of RNA such as the Cap structure at the 5′ end of tRNA molecules or a triphosphate group at the 5′ end of RNA polymerase I derived transcripts are not a problem in carrying out methods of the present invention. This is in contrast to many approaches to cDNA cloning and analysis that depend on modifications at the 5′ end as required, for example, for later amplification in a PCR reaction. According to certain protocols for the preparation of short RNA, a linker is ligated to the 5′ end of RNA [Harbers M., Genomics 91, 232-242 (2008)], Another example is the so-called oligo-capping method that specifically ligates RNA oligonucleotides to the 5′ end of full-length mRNA [Maruyama K. and Sugano S., Gene 138, 171-174 (1994)], Oligo-capping and other protocols used for short RNA cloning require an RNA-ligase-mediated modification of RNA molecules within the sample. This RNA ligation step is influenced by the last 5′-end nucleotides within the RNA molecule, which can lead to a biased representation during the analysis for molecules having different nucleotides at their 5′ end. Moreover, RNA molecules may require additional modification steps to create 5′ ends suitable for ligation of an oligonucleotide to the 5′ end of RNA. Such modifications include, but are not limited to, removal of a Cap structure by means of a pyrophosphatase, removal of phosphate groups by means of a phosphatase, and addition of a phosphate group to the 5′ end by means of a kinase. Thus the present invention represents a significant improvement over the prior art, since a simultaneous detection of all or most RNA molecules within a biological sample is not possible by other protocols that relay on modifications to the S′ end of RNA.

An aspect of the present invention requires modification of the 3′ end of RNA in a ligation or extension reaction, Either reaction allows for an extension of RNA molecules having free 3′- and 2′ ends, and an extension of RNA molecules having a free 3′ end and a blocked 2′ end in the last 3′-end nucleotides. For example, the cloning of piRNA with modified 2′ ends shows that 2′ end modifications do not necessarily interfere with reactions at the 3′ end.

Fragmentation of RNA can lead to RNA molecules that are phosphorylated at the 3′ end. If such fragmentation products are to be included into the analysis, the 3′ phosphoryl group has to be removed by means of a phosphatase including, but not limited to, the T4 polynucleotide kinase that has a 5′-kinase and 3′-phosphatase activity. For RNA molecules having blocked 3′ ends or 3′ and 2′ ends, other methods have to be applied, where, for example, the use of double-stranded adapters with partially or entirely random single-stranded overhangs can be applied (FIG. 1). Such an adapter may have blocked 3′ ends to avoid ligation to the 5′ end of RNA molecules within the sample at one oligonucleotide or both oligonucleotides. Nucleotides within the overhang could hybridize to regions at the 3′ end of RNA molecules forming a region of double-stranded DNA and RNA. For RNA having an open 3′ end, the double-stranded adapter can also be ligated to the last nucleotide by means of a DNA ligase. DNA ligases (EC 6.5.1.1) are commonly used to link double-stranded DNA strands; however, a single-stranded break within double-stranded DNA can be closed by a DNA ligase forming phosphodiester bond at the break point. To perform a ligation reaction, preferably phosphorylated 5′ end of one DNA strand reacts with an open hydroxyl group at the 3′ end of the other strand. Many DNA ligases are commercially available. For example, New England Biolabs and Fermentas provide such DNA ligases. Most commonly T4 DNA Ligase from bacteriophage T4, E. coli DNA ligase, or Taq DNA ligase are used for in vitro ligation reactions. A ligation step can prevent unspecific binding to sequences within RNA molecules. Otherwise, the overhang should be kept long enough to allow binding to the last or very 3′-end nucleotides and short enough to avoid binding to random sequences within RNA, and to allow bending off of the single-stranded DNA part from the double-stranded part within the adapter.

After hybridization, the double-stranded region is made of RNA from the RNA molecule within the sample and DNA from the adapter, and RNase H digestion can be used to remove a short stretch of RNA from the 3′ end of RNA molecules, RNase H (EC 3.1.26.4) is a commercially available enzyme (e.g. from New England Biolabs or Fermentas) that cleaves the 3′-O-phosphate-bond of RNA in DNA-RNA hybrids to produce termination products having a 3′-hydroxyl and 5′-phosphate group. Conditions for RNase H digestions are known to a person skilled in the art including, but not limited to, those disclosed in U.S. Pat. No. 6,544,741. The number of nucleotides removed from the 3′ end of RNA is determined by the length of the overhang of the adapter, and may be in the range of 1 to 4, 2 to 6, 4 to 10, or more than 10 nucleotides. Digestion of one or more nucleotides at the 3′ end of RNA can remove, at the same time, the last 3′-end nucleotides with a blocked 3′ end, and creating a new 37 end with an open 3′-hydroxyl group. Hence, the present invention provides the removal of blocked 3′ ends and blocked 3′- and 2′ ends from RNA molecules to make such RNA molecules available for modification of such 3′ ends. Thus the present invention provides the modification of any RNA species within an RNA sample to introduce a functional group for binding to a solid support as indicated in FIG. 2.

Introduction of a functional group at 3′ end of RNA molecules is the first step to modify RNA molecules for binding to a solid support. The binding to the solid support is facilitated by the interaction of a functional group at the 3′ end of RNA and a capturing group on the solid support. The functional group has high affinity to bind specifically to the capturing group on the solid support. The interaction between the functional group and the capturing group should be stable under all reactions performed on the solid support. The binding to the solid support may be reversible to release the RNA or any reaction products derived thereof. The functional group may be a compound such as biotin or digoxigenin binding to a high affinity capturing group on the solid support. In the case of biotin, the capturing group is made of avidin or streptavidin, in case of digoxigenin the capturing group is made of an anti-digoxigenin antibody. A person skilled in the art knows many more combinations of a functional group and a capturing group that can be used to bind an RNA molecule to a solid support including, but not limited to, chemical and high affinity binding reactions. Preferably, the functional group and the capturing group are made of polynucleotide where the functional group has a sequence complementary to the sequence of a capturing oligonucleotide attached to the solid support. More preferably, the functional group is made of oligonucleotide having sequences that are partially or entirely complementary to capturing oligonucleotide molecules attached on a solid support. Binding of modified RNA molecules to a solid support is achieved by hybridization of the functional group to a capturing oligonucleotide molecule bound to the solid support. The capturing oligonucleotide molecule on the solid support does not only capture RNA molecules for analysis, but at the same time, can function as a primer to drive a reverse transcription reaction.

The functional group at the 3′ end of RNA can be introduced in a tailing reaction (FIG. 3). In this embodiment, open 3′ ends of RNA are extended by adding homopolymers using a poly(A) polymerase [Huttenhofer A. et al., Methods Mol Biol 265, 409-428 (2004)]. Poly(A)polymerase or polynucleotide adenylyltransferase (EC 2.7.7.19) catalyzes the addition of adenosine to the 3′ end of RNA in a sequencoindependent fashion, but in vitro it can also incorporate other nucleotides as, for example, shown for C-tailing of RNA [Huttenhofer A. et al., Methods Mol Biol 265, 409-428 (2004)]. The enzyme is commercially available and can be purchased, for example, from New England Biolabs and Ambion. For the purpose of the present invention, homopolymers preferably include, but are not limited to, polyA or polyC chains. It is within the scope of the present invention to add in a first tailing reaction a polyA tail to the 3′ end of RNA molecules followed by a second tailing reaction to add a polyC tail to the last nucleotide of the polyA tail.

In a different embodiment, the first tailing reaction introduces a polyC tail to the 3′ end of RNA molecules, followed by a second tailing reaction to add a polyA tail to the last nucleotide of the polyC tail.

The present invention provides a method for modification of essentially any RNA molecules as the poly(A)polymerase reaction does not depend on the sequence of the RNA target molecule. Target RNA molecules may be non-polyadenylated RNA or polyadenylated mRNA. It is within the scope of the present invention to use C tailing rather than A tailing for modifying the 3′ end of RNA or otherwise to distinguish between polyadenylated and non-polyadenylated RNA.

According to a different embodiment, in a first reaction step, an oligonucleotide is ligated to the 3′ end of RNA followed by an extension reaction with a poly(A)polymerase. In this embodiment, modified RNA molecules derived from polyadenylated mRNA will have two polyA tails as compared to one polyA tail in modified RNA molecules derived from nonpolyadenylated RNA. The presence of one or more polyA regions may be used to distinguish between polyadenylated and nonpolyadenylated RNA. The length of the homopolymer added to the 3′ end of RNA may vary and depend on the reaction conditions. Preferably less than 50 nucleotides are added to the 3′ end of RNA molecules. Under different reaction conditions, more than 50 nucleotides are added to the 3′ end of RNA molecules. Extension of an RNA molecule in a polyadenylation reaction adds a polyribonucleotide chain to the 3′ end of RNA. Hence the last 3′-end nucleotide within the modified RNA has a diol group at the 3′ end. In one embodiment the diol group at the 3′ end is used for labeling. In a different embodiment, the 3′ end of RNA is labeled in a reaction using a terminal transferase, Reaction conditions for the 3′ end labeling of RNA by means of a terminal transferase are disclosed in U.S. Pat. No. 5,573,913.

In a different embodiment, oligonucleotides are added to the 3′ ends of RNA in a ligation reaction (FIG. 4). In the ligation step, an RNA adapter or an RNA-DNA hybrid molecule is commonly ligated to the open 3′ end of RNA using an RNA ligase (EC 6.5.1.3) [Michael M. Z., Methods Mol Biol 342, 189-207 (2006)]. The RNA ligase catalyzes an ATP dependent chemical reaction to link a 5′ phosphoryl-terminated nucleic acid donor molecule or an adapter molecule to a 3′ hydroxyl-terminated nucleic acid acceptor molecule or an RNA molecule within the sample, forming a phosphodiester bond between the two molecules. For the RNA ligation step, any RNA ligase can be used that can ligate a DNA and/or RNA oligonucleotide to RNA. Most commonly the T4 RNA ligase (for example, available from New England Biolabs and Fermentas) is used; alternatively, the Thermo Phage single-stranded DNA ligase can be used in this reaction. The Thermo Phage single-stranded DNA ligase is a commercially available enzyme that can work on both single-stranded DNA and RNA (for more information on the enzyme, refer to the product information at http://www.matis.is/prokaria/upload/files/Thermophage-ssDNA-ligase-version-4-2.pdf). The use of this enzyme may be preferable to directly ligating a DNA oligonucleotide to RNA. Hence the present invention provides a method for the direct ligation of DNA or RNA to an RNA molecule within the sample to prepare linear homopolymers composed of ribonucleotides, the RNA within the RNA sample and RNA portion of the adapter, or heteropolymers composed of ribonucleotides and desoxyribonucleotides, the RNA portion within the RNA sample and the DNA portion within the adapter.

In another embodiment, oligonucleotides may include portions made of RNA and DNA to form a hybrid molecule. Alternative approaches may also make use of double-stranded DNA adapters having an overhang made of single-stranded DNA as disclosed in WO2006003721, Double-stranded DNA adapters allow for a sequence-specific modification of selected RNA molecules specified by the sequence of the single-stranded overhang. The selected RNA molecules may be divided into polyadenylated and non-polyadenylated RNA. Ligation of a double-stranded adapter having an overhang made of single-stranded DNA to the 3′ end of RNA creates a molecule with partially double-stranded DNA-RNA and DNA-DNA hybrids. The oligonucleotide hybridizing to the RNA within the RNA-DNA portion at the 3′ end of the modified RNA molecules has to be removed to open the functional group for binding to the solid support. If the last 3-end nucleotide at the 3′ end of the modified RNA is made of a desoxyribonucleotide, there is no diol group at the 3′ end of modified RNA. In one embodiment, 3′ end is used for labeling. In a preferable embodiment, the 3′ ends of the adapter in one oligonucleotide or both oligonucleotides are blocked to avoid undesired ligation to the 5′ end of RNA.

In another embodiment, a double-stranded adapter with blocked 3′ ends is made of DNA and contains a recognition site for an endonuclease in the double-stranded region. After ligation of the adapter to RNA, the endonuclease is used to cut of the 3′ end region of the double-stranded adapter to create an open 3′ end at the 3′ end of the modified RNA. Since the endonuclease will only cut within double-stranded DNA, this digestion step will not cut the single-stranded RNA backbone within the RNA molecule.

In a different embodiment, the 3′ end of modified RNA is labeled in a reaction using a terminal transferase. Reaction conditions for the 3′ end labeling of RNA by means of a terminal transferase were disclosed in U.S. Pat. No. 5,573,913. This reaction does not require a diol group, but an open hydroxyl group at the 3′ end. Thus the terminal transferase mediated reaction can be used to label 3′ ends in RNA and DNA. An example labeling polyA-tailed DNA in a terminal transferase reaction with cyanine 3-ddTTP can be found in the supplementary information of Harris T D et al., Science 320, 106-109 (2008).

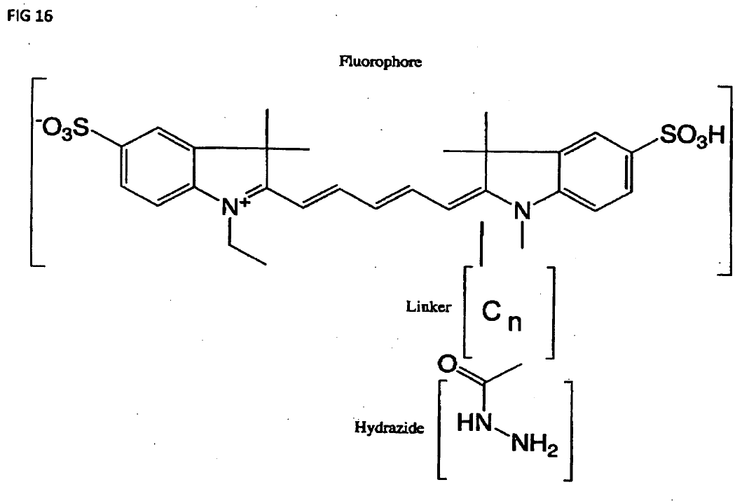

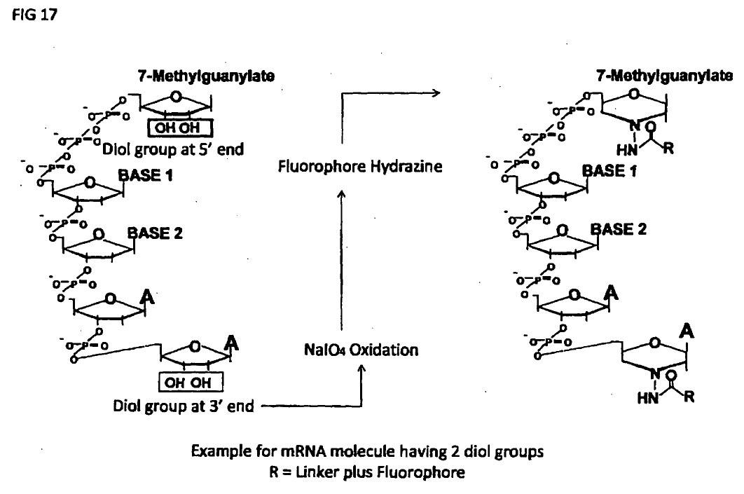

The present invention is not limited to the use of one specific nucleic acid molecule for the preparation of an adapter. A person skilled in the art will know many different ways for the preparation of DNA, RNA, or DNA-RNA oligonucleotides, DNA and RNA oligonucleotides are commercially available, for example, from Eurofins (http://www.eurofinsdna.com/products-services/oligonucleotides/dna-masynthesis.html). RNA and DNA oligonucleotide molecules can further be purchased, for example, from Invitrogen (http://www.invitrogen.com/content.cfm?pageid=9900), or Operon (http://www.operon.com/). Moreover, oligonucleotide molecules may be modified at their 5′ and/or 3′ end. Preferably, oligonucletide molecules used for adapter preparation are modified at their 3′ end to prevent ligation to the 5′ end of RNA molecules within the RNA sample (FIG. 5). Unfortunate ligation of oligonucleotide molecules to the 5′ end of RNA can lead to wrong sequencing results, when reads obtained from the 5′ end of RNA reveal adapter sequences rather than the desired sequence information from 5′ ends of RNA. For example, modified oligonucleotide groups can be obtained from Eurofins (http://www.eurofinsdna.com/products-services/oligonucleotides/modifications.html). Modification include, but are not limited to, a biotin or a digoxigenin group, reactive groups for cross linking or blocking like the 5′ aminolink C3/C5/C6/C12, 3′ aminolink C3/C6/C7, 3′ aminolink C3/C6/C7, amino (C2/C6)-dT, amino C6-dC, spacer C3/C9 (TEG), spacer C12/C18 (HEG), or a reduced thiol modifier, Oligonucleotide groups with blocked 3′ end do not have a diol group at their 3′ end. Hence DNA portions at the 3′ end of RNA/DNA hybrid oligonucleotide or RNA oligonucleotide molecules with blocked 3, end do no longer provide a diol groups for labeling at a later stage. The use of double-stranded adaptors provides an alternative approach to use oligonucleotide molecules with blocked 3′ ends that can be modified at a later stage as described in the foregoing. However, oligonucleotide groups ligated to the 3′ end of RNA molecules may carry a label (FIG. 9) or a functional group for binding to a solid support.

Different fluorescent dyes can be introduced as labels into synthetic oligonucleotides as for example commercially available from Eurofins [http://www.eurofinsdna.com/products-services/oligonucleotides/modifications/dna-modiflcations/fluorescent-dyes.html], or for example, by the use of a cross linking group at the 3′ end of the oligonucleotide. Alternatively fluorescent dyes can be introduced at the 3′ end of an oligonucleotide in a reaction mediated by a terminal transferase as described in the forgoing. Such a reaction can be used to label RNA or DNA oligonucleotides. Preferable fluorescent dyes for practicing the invention have been published by Ikeda S. and Okamoto A., Chem. Asian J. 3, 958-968 (2008). These fluorescence dyes are based on doubly-labeled nucleosides that are incorporated into oligonucleotide molecules. The absorption of the dye changes when shifting from the single-stranded DNA state to forming a double-stranded DNA in a hybridization reaction with a complementary nucleic acid molecule. Hence oligonucleotide molecules labeled in such manner can distinguish between single-stranded and double-stranded DNA. To practice the invention, oligonucleotide molecules labeled in such manner can be used in double-stranded adaptors to monitor the ligation of an adaptor to the 3′ end of RNA, to monitor the release of the oligonucleotide that hybridizes to the 3′ end of the RNA-DNA hybrid molecule, and/or to monitor the binding of modified RNA molecules to capturing oligonucleotide molecules on a solid support by means of a functional group contained in a labeled oligonucleotide. The label may remain bound to the solid support during all reactions performed on the solid support. Hence, labels as attached to desoxyribonucleotide may be used in a manner different from labels attached to ribonucleotide.

The oligonucleotide groups can be of different length. In one embodiment, the adapter is made of 10 to 25 nucleotides. In a different embodiment, the adaptor has 25 to 50 nucleotides. In just a different embodiment, the adaptor has 50 to 100 nucleotides, or has even more than 100 nucleotides. The oligonucleotides can be obtained by means of chemical synthesis or can be prepared by an enzymatic reaction. A person skilled in the art will know different DNA-dependent RNA polymerases such as the T3 RNA polymerase, T7 RNA polymerase, or SP6 RNA polymerase that can be used to prepare RNA molecules from a DNA template. Moreover, the oligonucleotide or its sequence can be partially or entirely of natural origin, it can be random, or it can be derived from a design process. During the design process, different parameters can be taken into account including, but not limited to, the cross hybridization to naturally occurring RNA and genomic DNA sequences, having specific nucleotides in their last 5′-end positions to improve the yield in a ligation reaction, having an A in the last 5′-end position, GC and AT content, the strength of the hybridization reaction to the capturing oligonucleotide on the solid support, the modification of the adaptor, the use of unnatural nucleotides or nucleotide analogs, the use of recognition sites for enzymes such as endonuclease, the introduction of an Identifier Sequence, be in part or over their entire sequence homopolymers, are composed in part or over their entire sequence of polyA or polyC, or composition of ribonucleotides and desoxyribonucleotides.

In a special embodiment the present invention makes use of oligonucleotides having Identifier Sequences. The Identifier Sequence may contain a recognition site for a restriction endonuclease to release double-stranded cDNA from the solid support. In a more preferable embodiment, the Identifier Sequence is located at the 5′ end of the oligonucleotide ligated to the 3′ end of RNA. Sequences within the Identifier Sequence are not complementary to sequences within the capturing oligonucleotide. Hence, sequences within the Identifier Sequence do not interfere with the capturing reaction to bind modified RNA to the solid support. Moreover, the sequence information within the Identifier Sequence can be obtained in a sequencing reaction driven by the capturing oligonucleotide on the solid support. Different Identifier Sequences may be used to practice the invention, where such sequence may be composed of 1 to 3, 3 to 6, or 6 to 10 nucleotides. Preferable Identifier Sequences are 1 to 3, 6 to 12, or 25 to 75 nucleotides in length. An Identifier Sequence may be of arbitrary nature; i.e., it can be a homopolymer, or may have a random sequence or a sequence designed by computational means. The Identifier Sequence can also be taken from a biological sample, may comprise recognition sites for restriction endonucleases or other enzymes including, but not limited to, DNA-dependent RNA polymerases or proteins including, but not limited to, those with affinity to binding to DNA or RNA, and it may have a sequence comprising priming sites, or a target sequence for a probe that can hybridize to the Identifier Sequence. It can also be artificially created. Identifier Sequences can be designed in accordance to any or all for the following rules:
The last 5′-end nucleotide improves the yield of an RNA ligase reaction.
The last 5′-end nucleotide is an A to improve the yield of an RNA ligase reaction.
Be of sufficient length to enable identification by means of sequencing or hybridization.
Sequences of different Identifier Sequences used within the same experiment must be distinct.
Different Identifier Sequences used within the same experiment should be distinct in more than one position to enable a clear identification even where sequencing errors occur within the Identifier Sequence.
Identifier Sequences should avoid sequences having a structure or sequences that may interfere with the sequencing reaction (e.g. G-rich sequences, or palindromes).
Identifier Sequences may be selected to form stable hybrids with complementary sequences.
Identifier Sequences may have sequences that enable specific manipulations or binding to dedicated proteins, e.g. restriction endonucleases, DNA dependent RNA polymerases, DNA binding proteins, RNA binding proteins, or transcription factors.
Identifier Sequences should avoid sequences that may interfere with the manipulation of RNA and DNA.

If Identifier Sequences are introduced in the first reaction step, individual samples within a pooled sample are mixed at the earliest possible stage. It is preferable to introduce the Identifier Sequence at an early stage prior to binding the RNA molecules to a solid support. However, it is within the scope of the present invention to introduce an Identifier Sequence or even a second Identifier Sequence at a later stage. In a preferred embodiment, an Identifier Sequence is introduced prior to binding to the solid support. In a different embodiment, the Identifier Sequence is introduced with the adaptor ligated to the 3′ end of cDNA molecules attached to the solid support prior to obtaining sequences from the 5′ end of RNA.

In just a different embodiment, two Identifier Sequences may be used. The first Identifier Sequence is introduced prior to binding of the RNA molecule to the solid support, and the second Identifier Sequence is introduced prior to obtaining sequence information from the 5′ end of RNA. An Identifier Sequence can further be introduced for a selective modification of RNA molecules within a sample by ligation of an oligonucleotide to the 5′ end. Approaches toward the selective modification of 5′ ends within RNA have been described in the literature, for example, for the so-called oligo capping method [Maruyarna K. and Sugano S., Gene 138, 171-174 (1994)], or otherwise disclosed in US Patent Application Publication No. 2008/0108804.

Identifier Sequences are used to mark the origin of a sample within pools of different samples. In one embodiment, the Identifier Sequences are used to mark particular RNA molecules within a biological sample for an experiment which makes use of multiple biological samples including, but not limited to, those taken from different organisms, tissues, cell types, treatments, or various stages of a biological experiment. The pooling of samples within an experimental design may facilitate different functions including, but not limited to, cutting costs, enabling simplified handling of many samples by reducing the number of samples to be handled at the same time, increasing the complexity of the sample to make full use of the very high throughput sequencing approaches (so-called multiplex sequencing [Church G. M. and Kieffer-Higgins S., Science 240, 185-188 (1988), U.S. Pat. No. 4,942,124]), or enabling certain forms of data analysis. In one preferable embodiment, samples in a pool are pooled to have the same systematic error over all steps of the manipulation for a common statistical analysis [US Patent Application Publication No. 2009/0108803]. Hence the present invention provides for the introduction of different Identifier Sequences to different RNA samples or molecules in separate ligation or extension reactions, so as to pool the reaction products to prepare a pooled RNA or DNA sample for analysis. Within the pooled RNA sample, the origin of each sample or RNA molecule can be identified by reading out the sequence within the Identifier Sequence.

In a different embodiment, Identifier Sequences can be used to specifically capture modified RNA by means of hybridization to a capturing oligonucleotide having complementary sequence to the Identifier Sequence, or use of specific sequencing primers that hybridize to Identifier Sequences to drive selective sequencing reactions. Hence, different RNA molecules or RNA samples within a pool of RNA samples can be separated by binding to different solid supports or different locations on the same solid support. Selective hybridization reactions to Identifier Sequences are preferable to read out Identifier Sequences attached at the 3′ end of cDNA on a solid support or the 5′ end of RNA.

In one embodiment, the present invention makes use of different Identifier Sequences attached to the 5′ end of RNA as disclosed in US Patent Application Publication No. 2008/0108804 to prime selectively the sequencing of 5′ end of RNA molecules carrying different modifications at the 5′ end. In a preferable embodiment, the 5′ modification is a Cap structure. In a different embodiment, the Identifier Sequence is used to distinguish between RNA molecules having distinct modifications at the 5′ end. In just a different embodiment, functional groups added to the 3′ end of RNA may be comprised of regions for binding to the solid support and regions that function as an Identifier sequence, which functions as a priming site to drive the sequencing reaction. Hence different RNA molecules or RNA samples within a pooled of RNA samples can be distinguished by the use of different sequencing primers driving sequencing reactions to obtain sequence information from the 3′ end and/or 5′ end of RNA. In a preferable embodiment, Identifier Sequences are identified by sequencing.

In a different embodiment, Identifier Sequences function as selective priming sites to drive independent sequencing reactions. In just a different embodiment, Identifier Sequences contain recognition sites for endonucleases that can be used to selectively modify groups of RNA molecules within a pool of derived RNA molecules or cDNA molecules. Such recognition sites for endonucleases may be used to release double-stranded cDNAs from the solid support. In just a different embodiment, Identifier Sequences contain promoter regions for DNA-dependent RNA polymerases that can be used to selectively prepare new RNA copies of the cDNA molecules attached to the solid support. Preferably promoter regions for DNA dependent RNA polymerases are introduced at the S′ end of RNA, equal to the 3′ end of single-stranded cDNA on the solid support. RNA synthesis by a DNA-dependent RNA polymerase from a template DNA attached to a solid support may be used to amplify RNA within a biological sample. Amplified RNA may be used for further analysis including re-sequencing by means of the present invention or otherwise by sequencing in a shotgun approach to obtain sequence information from Internal regions of such RNA molecules.

For analysis, RNA molecules are labeled in a specific manner. Preferably the label is introduced in a reaction that labels only RNA molecules, but not DNA molecules. DNA molecules have to be excluded from the labeling reaction to assure that DNA contaminations within RNA samples are not included into the analysis. Hence, the labeling reaction is an important quality control step as needed to distinguish between RNA molecules and DNA molecules. For the analysis of RNA molecules and sequences derived thereof it is an absolute requirement to exclude DNA from the analysis that can lead to misleading results. In particular, genomic DNA contaminations are a problem to the data analysis as sequences derived from genomic DNA will map to genomic sequences used in the data analysis, and as such lead to wrong results on potentially transcribed regions. In a different embodiment, labels attached to the RNA molecules are used to confirm binding of modified RNA molecules to a solid support. To reliably detect the position of an RNA molecule on the solid support is an important quality control step to monitor an effective loading of the solid support prior to starting the analysis of molecules attached to the solid support. Moreover, it is important to mark the positions on the solid support where modifications of an RNA molecule occur. Binding to the solid support allows performing many reaction steps on the immobilized molecule, where the label can prove that the molecule stayed on the solid support at all times during the entire process. Such controls are in particular needed, where sequences are obtained in multiple reactions, and the final sequence information is assembled from sequence information obtained from a defined location on the solid support.

In another embodiment, specific RNA molecules within the RNA sample are selectively labeled. Such a labeling reaction can be used to measure the portion of certain RNA molecules within the sample, or otherwise can have specific functions in monitoring reactions on the surface of the solid support. The label may function as an Identifier Sequence within a pooled sample. In a different embodiment, the label may be used for an internal control. Hence the labeling strategy of the inventions provides various means to perform different quality control steps during the analysis of RNA molecules on a solid support.

RNA molecules are distinct from DNA molecules by the presence of a terminal diol group in the last 3′-end nucleotide. In addition, mRNA molecules are modified at their 5′ end by the Cap structure. This Cap structure introduced a second diol group that is specific for full-length mRNA molecules (FIG. 17). Different reactions are described in the literature to modify diol groups within RNA in a chemical reaction [refer to Proudnikov D. and Mirzabekov A. Nucleic Acid Res. 22, 4535-4542 (1996) and reference cited in there]. For example, the diol group in RNA can be converted into dialdehyde groups by oxidation [Czworkowski J et al., Biochemistry 30(19):4821-30 (1991); Odom O W Jr et al., Biochemistry 19(26):5947-54 (1980)]. Most commonly sodium periodate is used for oxidation reaction, Periodate-mediated oxidation of vicinal diol groups provides one of the few methods to selectively modify the 3′-terminal ribose in RNA molecules. Thereafter, periodate-oxidized ribonucleotides can be subsequently converted to fluorescent nucleic acid molecules by reaction with fluorescent hydrazines, hydroxylamines and amines [Hileman R E et al., Bioconjug Chem, 5(5):436-44 (1994)]. Preferably the dialdehyde groups in periodate-oxidized ribonucleotides can be used to introduce a label in a second chemical reaction with a hydrazine group. Reaction conditions for the modification of diol groups within RNA in a chemical reaction are disclosed in U.S. Pat. Nos. 5,962,272 and 6,022,715. In one embodiment, the labeling reaction is performed directly on an RNA sample. In a different embodiment, the labeling reaction is performed on modified RNA. In just a different embodiment, the labeling reaction is performed on RNA immobilized on a solid support. It is further within the scope of the invention to perform more than one labeling reaction at different time points during the analysis, where a label may be removed during the course of the analysis.

The label is most preferably a fluorescent dye. Fluorescent dyes can be detected in real time with high resolution, and the availability of many fluorescent dyes with distinct excitation and emission wavelengths allow monitoring many labels in one experiment. Preferably, sets of fluorescent dyes are selected so as to allow for a simultaneous detection of more than one dye in the same reaction. A set of dyes that can be detected at the same time include, but are not limited to, Cy3, Cy5, FAM, JOE, TAMRA, ROX, dR110, dR6G, dTAMRA, dROX, or any mixture thereof (refer to Table 2 below for details on those dyes). Any of those dyes may be used individually or in any combination to practice the present invention. More preferably, a dye should allow for single molecule detection. Examples for the use of fluorescence methods in single molecule detection have been described by Joo C et al., Annu Rev. Biochem. 77, 51-76 (2008). A large number of fluorescent dyes has been synthesized, and are commercially available in different formats [refer to http://www.analytchem.tugraz.at/fluorophores/ and http://en.wikipedia.org/wiki/Category:Fluorescent_dyes for examples on different fluorescent dyes]. This includes fluorescent dyes having a linker region and a hydrazine group (refer to FIG. 16 for an example structure) that allows for coupling to RNA in a reaction with dialdehyde groups. For examples on such compounds refer to the catalog of Invitrogen [http://probes.invitrogen.com/servlets/pricelist?id=32084]. The present invention is not limited to the use of a specific fluorescent dye, but different dyes can be applied to the same effect. The linker region may consist of a carbon backbone, may contain sulfur atoms, ketone groups, or diethylene glycol groups, or dodecaethylene glycol groups. The length of the linker can vary where the backbone is a linear molecule of 1 to 20 atoms. Preferably, the linker has more than 10 atoms; more preferably the linker has more than 15 atoms. A linker may contain groups of atoms that allow for selective removal of the label in a chemical reaction as, for example, disclosed in PCT Patent Publication No. WO2003/048387,

TABEL 2
Use of different fluorophores

| Dye | Excitation | Emission |
|---|---|---|
| Cy3 | 530 | 570 |
| Cy5 | 650 | 670 |
| FAM | 490-495 | 515-520 |
| JOE | 520-525 | 550-555 |
| TAMRA | 550-555 | 580-585 |
| ROX | 580-585 | 605-610 |
| dR110 | 519 | 539 |
| dR6G | 547 | 567 |
| dTAMRA | NA | 590-595 |
| dROX | NA | 615-620 |

NA = No information in literature

The invention can make use of one or more fluorescence dyes, where the detection of a fluorescence dye can be a one-time event or can be repeated at different time points. Preferably, different fluorescence dyes that can be detected independently are used. Table 2 provides examples of different fluorophores having distinct emission wavelengths as needed for parallel detection in a multi color reaction. Different fluorophores can be used for the detection of:
Binding of RNA molecules to a solid support by labeling the 3′ end of RNA.
Binding of an extended cDNA molecule to the solid support by addition of a label to the 3′ end of single-stranded cDNA,
Each of the 4 nucleotides within RNA or DNA detected by use of the same dye.
Each of the 4 nucleotides within RNA or DNA detected by use of more than one dye.
Each of the 4 nucleotides within RNA or DNA detected by use of four different dyes.
The Cap structure in full-length mRNAs.
Labeling of groups of RNA molecules derived from the same sample within a pool of different samples.
Use of internal controls.

Hence, the present invention can make use of one dye to monitor diol groups within RNA, two dyes for monitoring the diol groups at 3′ end of RNA and the diol groups within the Cap structure of mRNA, three dyes for monitoring diol groups within RNA and each of the 4 nucleotides within RNA or DNA by use of the same dye, four dyes for monitoring diol groups within RNA and each of the 4 nucleotides within RNA or DNA by use of more than one dye or even four different dyes, provided that the one dye is used for labeling the diol groups and one nucleotide, five dyes for monitoring diol groups within RNA and each of the 4 nucleotides within RNA or DNA using distinct dyes, six dyes for monitoring the diol groups at 3′ end of RNA, the diol groups within the Cap structure of mRNA, and each of the 4 nucleotides within RNA or DNA using distinct dyes, six dyes for monitoring the diol groups at 3′ end of RNA and the diol groups within the Cap structure of mRNA (by the same dye), each of the 4 nucleotides within RNA or DNA using distinct dyes (four dyes), and an Identifier Sequence (one dye), or seven dyes for monitoring the diol groups at 3, end of RNA and the diol groups within the Cap structure of mRNA (two dyes), each of the 4 nucleotides within RNA or DNA using distinct dyes (four dyes), and an Identifier Sequence (one dye). An Identifier Sequence may be placed at the 3′ end of RNA or the 3′ end of cDNA. Examples for the use of different dyes in sequencing reactions can be found in Sensen C. W., Essentials of Genomics and Bioinformatics, Wiley-VCH, Weinheim 2002, page 165. RNA molecules may carry more than one fluorescent dye. For example, RNA molecules with a free 3′ end may carry one fluorescent dye whereas RNA molecules having a Cap structure and a free 3, end may carry two fluorescent dyes. Hence different RNA molecules can have different signal strength in the detection reaction. Such difference in the single strength can be used to distinguish between RNA molecules carrying one label and RNA molecules carrying two labels. Hence the detection of a fluorescent dye can be used to distinguish between different RNA molecules and to specifically detect mRNA molecules among other RNA molecules. In this embodiment, the present invention can be used to determine the mRNA content of an RNA sample. In a different embodiment, the present invention can be used to determine cDNA molecules that have been extended to the 5′ end of mRNA molecules contained in an RNA sample.

In a different embodiment, distinct dyes are used to mark internal controls and monitor them during the process. Preferably, internal controls are added to the RNA sample prior to beginning the experiment. Hence, an internal control can be used to monitor the yield of each reaction step, the binding to a solid support, and the presence on the solid support. Moreover, internal controls can have specific features to monitor reaction conditions for specific RNA molecules. Such features include, but are not limited to, the length of an RNA molecule, the G/C and A/T content, the ability to form secondary structures, whether their sequences are distinct from other sequences used in the experiment, whether they have unique sequences, whether they hybridize to other RNA molecules within the sample, or whether they have a functional group at the 5′ or 3′ end, contain an Identifier Sequence, or carry a modification. Preferably, internal controls are prepared from a template. Such a template includes, but is not limited to, a PCR product, a PCR product having ends to enable an in vitro transcription reaction, a synthetic DNA or RNA, a plasmid, or a cloned cDNA. More preferably, an internal control is prepared from a cDNA cloned into a vector having features to enable in vitro transcription. The preparation of the internal control from a cloned cDNA template comprises the following steps:
An RNA molecule of defined sequence and length is prepared by in vitro transcription reaction.
Optionally, a Cap structure is introduced to the RNA transcript during the in vitro transcription reaction.
Optionally, the RNA molecule may be modified at the 3′ end in a ligation or extension reaction.
Optionally, the ligation or extension reaction is carried out in order to introduce a functional group.
Optionally, the ligation or extension reaction is carried out in order to introduce a label.
Optionally, a label is introduced in a chemical reaction using one or two diol groups within the RNA molecule.
Optionally, a label is introduced in an enzymatic reaction using a terminal transferase to modify the 3′ end within the RNA molecule.
Purification of modified RNA molecules.
Quantification of modified RNA molecules.

A person skilled in the art knows standard reaction conditions for these steps. Different DNA-dependent RNA polymerases such as the bacteriophage T3 RNA polymerase, T7 RNA polymerase, or SPF6 RNA polymerase are commonly used for in vitro transcription of cDNAs cloned into vectors having promoter sites for one or more of those RNA polymerases or PCR products having such promoter sites. These DNA-dependent RNA polymerases initiate transcription from specific double-stranded promoters, and the in vitro transcription reaction can be terminated, for example, by linearizing the template. RNA is synthesized in the 5′ to 3′ direction and can make use of single-stranded DNA or double-stranded DNA templates having a prober promoter sequence. T3 RNA polymerase, T7 RNA polymerase, and SP6 RNA polymerase are commercially available, for example, from Fermentas. An example of a protocol for the preparation of RNA by means of RNA polymerase can be found on the homepage of Fermentas under http://www.fermentas.com/techinfo/modifyingenzymes/protocols/p_synthstrspecrna.htm.

Optionally, a Cap structure can be introduced during the in vitro translation reaction. For example, the Cap analog [m⁷G(5′)ppp(5′)G] can be added to RNA polymerase reactions for the synthesis of 5′ capped RNA molecules in in vitro transcription reactions. The compound can be commercially obtained from Ambion. The introduction of a Cap structure at the 5′ end of an internal control can be preferable to monitor extension reactions on the solid support. RNA molecules obtained by in vitro transcription can be purified by DNase treatment to remove the DNA template. Other purifications steps as commonly used in the field may be used as well to remove the RNA polymerase and nucleotides used in the transcription reaction.

Optionally, the purified RNA molecule may be forwarded to a ligation or extension reaction as described in the forgoing. It is within the scope of the present invention that an internal control is modified by any of the aforementioned methods to introduce a functional group or a label.

In a preferred embodiment, an internal control is modified to introduce a label as described in Ikeda S. and Okamoto A., Chem. Asian J. 3, 958-968 (2008). Purified RNA molecules can be then labeled, where a fluorescent dye is introduced for chemical reaction with the one or two diol groups within the RNA molecules. This reaction is preferably carried out under the conditions disclosed in U.S. Pat. Nos. 5,962,272 and 6,022,715. Alternatively, the RNA molecule can be labeled in in vitro reaction using a terminal transferase under the conditions disclosed in U.S. Pat. No. 5,573,913. After the labeling reaction, the labeled RNA molecules are purified of free dye. The purification step may be done by the use of commercial products for RNA and DNA purification by chromatography or gel filtration. Such products are, for example, commercially available from Millipore, GE Healthcare, or Qiagen. Preferably, labeled RNA molecules are quantified prior to their use in an experiment.

Various methods for quantification of RNA are known to a person skilled in the art, or can otherwise been found in Sambrook J. and Russuell D. W., Molecular Cloning, A Laboratory Manual, Cold Spring Harbor Laboratory Press, New York, 2001. More preferably, internal controls are added to RNA samples at a defined concentration. Even more preferable, the number of molecules representing internal controls within an RNA sample is known. Labeled RNA molecules or internal controls can be used for other purposes independently from their use to conduct the invention. Even more preferable internal controls are labeled at the ends to avoid any interference of the labels in primer extension reaction such as a reverse transcription reaction, a DNA synthesis, an RNA sequencing reaction, a DNA sequencing reaction, or an amplification reaction. Even more preferable, internal controls can be monitored independently from other molecules within the sample. Even more preferable, internal controls have a sequence distinct from RNA molecules contained within a sample. Hence internal controls can be directly added to a biological sample during RNA preparation, and the recovery of the internal control is done by sequencing in the cause of the experiment. Hence internal control may have sequences not found in genomic sequences. In a different embodiment, the internal control is unlabeled and added to the RNA sample without having a label. In this embodiment, the internal control may become labeled while practicing the present invention. Internal controls are identified by sequencing, and their sequences can be related to their position on the solid support, contribution of internal controls to the total number of RNA molecules within the sample or experiment, full-length ratio of cDNAs, and/or recovery rate of the process.

Modified RNA molecules and/or RNA molecules intended as internal controls are captured and bound to a solid support for further manipulation and analysis. The binding to the solid support may be facilitated by various means known to a person skilled in the art including, but not limited to, the examples in US Patent Application Publication No. 2008/0108804. Such RNA molecules may carry a label or can be free of any label. For binding to the solid support, the capturing oligonucleotide attached to the solid support is made of oligo-dT to bind to polyA tails or is made of oligo-dG to bind to polyC tails. A polyadenylated mRNA molecule may bind directly to the solid support by hybridization of its polyA tails to an oligo-dT oligonucleotide on the solid support. In a different embodiment, the mRNA molecules having a polyA tail bind to oligo-dT oligonucleotides on the solid support whereas non-polyadenylated RNA molecules bind to the solid support by means of a functional group that does not have polyA. Hence, the invention provides a method for separating polyadenylated RNA from other RNA species on the solid support.

In a different embodiment the capturing oligonucleotide has a sequence partially or entirely complementary to the functional group or an oligonucleotide ligated to the 3′ end of RNA molecules. In a preferable embodiment, the capturing oligonucleotide has at its 3′ end a sequence complementary to the functional group it binds to. Hence, the capturing oligonucleotide can function as a primer to drive a primer extension reaction and/or a sequencing reaction.

In a different embodiment, the capturing oligonucleotide has at its 5′ end a sequence that is not complementary to any sequence within the functional group. In this embodiment, the RNA molecule binding to the solid support has sequences at its 3′ end that do not bind to the capturing oligonucleotide on the solid support, and as such remain as single-stranded RNA or DNA after binding to the solid support. The sequence and length of the capturing oligonucleotide or the region within the capturing oligonucleotide that binds to the functional group within the RNA molecule defines the strength of the binding reaction. Sequences of the capturing oligonucleotide may be selected based on their GC or AT content. More preferably, sequences of the capturing oligonucleotide are selected be specific for the binding reaction to the functional group, and do not bind unspecific to sequences contained in RNA molecules within the sample.

A capturing oligonucleotide may be 10 to 15 nucleotides long, 15 to 20 nucleotides long, 20 to 30 nucleotides long, 30 to 40 nucleotides long, or longer than 40 nucleotides. Preferable capturing oligonucleotides have a length of 20 to 40 nucleotides. A capturing oligonucleotide can be made of DNA, modified DNA, RNA, modified RNA, peptide nucleic acid (PNA) having a nucleic acid with a peptide-bond backbone, locked nucleic acid (LNA) having ribose moieties with an extra bridge connecting the 2′ and 4′ carbons, or any mixture thereof.

After binding the RNA molecules to a solid support, certain reactions may be performed on such immobilized molecules bound to the solid support at defined locations. The location of immobilized molecules may be determined by holes or depressions provided in the solid support that function as reaction compartments, by a printing process that places capturing oligonucleotides on the solid support in an arrayed format, or by different capturing oligonucleotides positioned at different locations. Immobilized molecules may also be entirely randomly located on the solid support surface. If the solid support is made of beads, the beads may be placed randomly in a flow cell, located in depressions, or otherwise grouped by physical properties. Beads may carry a label of their own, where such a label includes, but is not limited to, an electric charge, a spin, fluorescence, a magnetic momentum, a quantum dot, or any mixture thereof.

Binding of modified RNA molecules to the solid support are monitored by detection of a label attached to the RNA molecule.

The processes discussed below are described for individual RNA and DNA molecules, but they apply equally to any RNA and DNA molecules attached to the solid support. However, some of the reaction steps may be specific to certain RNA molecules, and as such they may apply only to some of the RNA or DNA molecules on the solid support. Also, the reactions discussed below can be consecutively performed and certain reaction steps may not involve all RNA or DNA molecules on the solid support, and some of the molecules on the solid support may remain unchanged in certain reactions. The reactions are performed simultaneously in a parallel manner on all RNA or DNA molecules bound to the solid support. In one embodiment, the reaction conditions are the same at all locations on the solid support. In a different embodiment, the solid support is divided into different compartments. The reaction conditions may be the same in all compartments on the solid support, or different reaction conditions may be use in different compartments on the solid support Different compartments may be further use to separate groups of RNA molecules according to common feature, distinct features, the functional group at the 3′ end, or the origin of the RNA sample. Preferably, most or all reactions on the solid support are performed in the presence of an additive. Such additives include but are not limited to trehalose, glucose, sorbitol, betanin, or any mixture thereof. Most preferably the additive is D (+) trehalose.

In a first reaction step on the solid support, the capturing oligonucleotide is used to prime a reverse transcription reaction. RNA molecules attached to the solid support may be used as templates to prepare a DNA transcript, a so-called cDNA, by means of a reverse transcriptase. A person skilled in the art knows many modifications of this process including different reaction conditions and enzyme modifications including those described in Sambrook J. and Russuell D. W., Molecular Cloning, A Laboratory Manual, Cold Spring Harbor Laboratory Press, New York, 2001. Preferably, the reverse transcription reaction is performed in the presence of trehalose under conditions disclosed in U.S. Pat. No. 6,013,488 and US Patent Application Publication No. US2001/0012617. Reverse transcriptases include, but are not limited to, the HIV-1 reverse transcriptase, AMV reverse transcriptase, M-MLV reverse transcriptase, M-MLV reverse transcriptase RNase H minus, or any other modifications thereof. It is also within the scope of the invention to use any mixture of different reverse transcriptases. Reverse transcriptases are commercially available from different providers including, but are not limited to, Invitrogen, Promega, Fermentas, Epicentre and others offering for example M-MuLV Reverse Transcriptase, H Minus M-MuLV Reverse Transcriptase, Superscript II, Superscript III, AMV Reverse Transcriptase, MonsterScript, Expand Reverse Transcriptase.

A first reverse transcription reaction can be used in:
Fill-in reaction to extend homopolymers at the 3′ end of RNA molecules until the end of the RNA molecule is reached (FIG. 10). Homopolymers include, but are not limited to, polyA tails at the 3′ end of RNA hybridizing to oligo-dT sequences on the solid support. Providing only dTTP in the reaction mixture, the reaction will terminate at the first nucleotide within the RNA that is not an “A”. Hence, the fill-in reaction will terminate at the 3′ end of the RNA. Similarly, any other homopolymer made of polyC, polyG, or polyT may be added to the 3′ end of RNA and used in the hybridization and fill-in reactions.
Fill-in reaction to synthesis a cDNA having complementary sequence to the RNA template (FIG. 14). In this embodiment, the reaction conditions allow for extending the cDNA up to the 5′ end of the RNA. In this embodiment standard reaction conditions for the synthesis of a cDNA on a solid support can be used. Preferably, the reverse transcription reaction is performed in the presence of trehalose under conditions disclosed in U.S. Pat. No. 6,013,488 and US Patent Application Publication US2001/0012617.

Sequencing reaction to obtain sequence information from the 3′ end of RNA (FIG. 11). In this reaction, the sequence of a number of nucleotides is determined, where the number of nucleotides may be smaller than number of nucleotides in the RNA template. Different protocols for the sequencing of RNA by means of a reverse transcriptase have been published in the literature including, but not limited to, Koper-Zwarthoff E. C. et al., Nucleic Acid Res. 7, 1887-1900 (1979) and Bauer G. J., Nucleic Acid Res. 18, 879-884 (1990). To practice the invention, RNA sequencing is preferably conducted by a sequencing-by-synthesis method that detects the incorporation of each nucleotide. The incorporation reaction may detect 4 different nucleotide having 4 different dyes at the same time, or 4 different nucleotides are tested for incorporation in separate reactions. Different methods for sequencing-by-synthesis for DNA sequencing have been described in the literature and are commercially available, for example, from 454, now ROCHE, Illumina, or Helicos BioSciences. The same chemistry may be used to sequence RNA by means of a reverse transcriptase. Such approaches may make use of labeled nucleotides to monitor the incorporation of such a labeled nucleotide in an extension reaction (Illumina and Helicos), or otherwise monitor reaction products derived from an extension reaction, such as pyrophosphate molecules cleaved from the incorporated nucleotide (454 and Pyrosequencing). In an alternative embodiment, the sequencing is conducted using a hybridization approach.
Fill-in reaction to extend cDNAs derived from a sequencing reaction to reach the 5′ end of the RNA templates (FIG. 11). In this embodiment standard reaction conditions for the synthesis of a cDNA on a solid support can be used. Preferably, the reverse transcription reaction is performed in the presence of trehalose under conditions disclosed in U.S. Pat. No. 6,013,488 and US Patent Application Publication No. US2001/0012617.

After the reverse transcription reaction step, molecules attached to the solid support are composed of an RNA template and a cDNA strand having complementary sequence to the entire RNA template or parts of the RNA template. Regions of single-stranded RNA may remain at the 3′ end of RNA if they are not complementary sequences within the capturing oligonucleotide. These regions remain single-stranded because they do not hybridize to the capturing oligonucleotide. In one embodiment, the functional group is designed in such a way that the last 3′end nucleotides do not bind to the capturing oligonucleotide. Hence the present invention provides a method for keeping regions of single-stranded RNA at the 3′ end of RNA templates. Other regions of RNA may remain single-stranded because the reverse transcription reaction may fail to reach the 5′ end of an RNA template. In particular, for the synthesis of a cDNA from long mRNAs, there is a risk that for some of the molecules the reverse transcriptase fails to reach the 5′ end of the RNA template.

Regions of single-stranded RNA can be removed from the cDNA-RNA hybrids by treatment with RNase I that specifically digests single-stranded RNA. RNase I is a commercially available enzyme that can, for example, be obtained from New England BioLabs or Fermentas. Digestion with RNase I can be used to specifically detect full-length cDNA-RNA hybrids having a Cap structure under similar conditions as used for the Cap-Trapper full-length cDNA cloning method [Carninci P. and Hayashizaki Y., Methods Enzymol. 303, 1944 (1999), and U.S. Pat. Nos. 5,962,272 and 6,022,715]. In this embodiment, an mRNA molecule caries a label at the 3′ end and at the Cap structure at the 5′ end. Treatment with RNase I will remove all regions made of single stranded RNA. During RNase I digestion labels attached to the 3′ end can be removed. In addition, RNase I will remove the label from 5′ ends of those cDNA-mRNA hybrids that have not been extended to the 5′ end of the mRNA template. Hence, only labels attached to the Cap structure of full-length cDNA-mRNA hybrids will remain on the solid support for detection. Thus the invention provides a method for detecting full-length cDNA in cDNA-mRNA hybrids derived from mRNA. In one embodiment, the full-length cDNA is detected on a solid support. In a different embodiment, the full-length cDNA is detected in solution.

In just a different embodiment, the full-length cDNA is derived from an internal control. In just a different embodiment, the label at the 3′ end is not removed because the functional group and the capturing oligonucleotide have complementary sequence over the entire sequence of the functional group, or because the label at the 3′ end was not attached to a diol group at the 3′ end. In case the label remains at the 3′ end while detecting labels attached to the Cap structure, full-length cDNAs may be detected by a stronger signal strength caused by two instead of one labeling group within an RNA molecule, or may be detected by the use of distinct labels added to the Cap structure and to the 3′ end and/or the functional group.

The cDNA-RNA hybrids are attached to the solid support by means of the capturing oligonucleotide. Hence the cDNA strand is attached to the solid support, whereas the RNA strand can be removed for example by heat treatment alkali treatment or digestion by RNase H. RNase H digestion may be used to introduce priming sites for second strand synthesis [Gubler U. and Hoffman B. J. Gene 25, 263-269 (1983)] or random sequencing in a shotgun like approach. After removal of the RNA template, the cDNA molecules on the solid support are modified at their 3, end to introduce a priming site (FIG. 12). The priming site can be made of an oligonucleotide ligated to the 3′ end or can be made of a homopolymer added in an extension reaction. Preferably, a double-stranded DNA adapter having a single-stranded overhang is ligated to the 3′ end of cDNA. Such an overhang can be 4 to 6 nucleotides in length, 6 to 8 nucleotides in length, 8 to 12 nucleotides in length, or more than 12 nucleotides in length. A person skilled in the art knows different approaches using partly double-stranded oligonucleotides in a ligation reaction as for example published by Shibata Y. et al. in Biotechniques, 30, 1250-1254 (2001) and US Patent Application Publication No. US2004/166499. DNA adapters are ligated to the cDNA by means of a DNA ligase. DNA ligases (EC 6.5.1.1) are commonly used to link double-stranded DNA strands. Many DNA ligases are commercially available for example from New England Biolabs and Fermentas, where most commonly T4 DNA Ligase from bacteriophage T4, E. coli DNA ligase, or Taq DNA ligase are used for in vitro ligation reactions. In one embodiment, the overhang has a random sequence; in a different embodiment the overhang has a defined sequence for targeting of specific RNA molecules.

In a preferred embodiment, the single-stranded overhang within a double-stranded adapter is made of oligo-dA and the double-stranded adapter with a single-stranded oligo-dA overhang is use to block the 3′ end of capturing oligonucleotide on the solid support made of oligo-dT. In another preferred embodiment, the single-stranded overhang within a double-stranded adapter has complementary sequence to the sequence of the capturing oligonucleotide on the solid support. The double-stranded adapter with a single-stranded overhang complementary sequence to the sequence of the capturing oligonucleotide on the solid support is use to block the 3′ end of capturing oligonucleotide on the solid support. Hence the invention provides means to block the 3′ end of those capturing oligonucleotides that did not hybridize to an RNA molecule, or of capturing oligonucleotides, for which now primer extension reaction occurred. Blocking the 3′ end of such capturing oligonucleotides can be done at different time points. Preferably blocking the 3′ end of such capturing oligonucleotides is performed after the RNA templates have been removed from the solid support. Blocking the 3′ end of such capturing oligonucleotides can prevent obtaining useless sequence information from capturing oligonucleotides rather than 5′ ends of RNA.

In just a different embodiment, the adaptor introduces an Identifier Sequence, a promoter for a DNA dependent RNA polymerase, a recognition site for a restriction endonuclease, or a label. A label may be contained in one or more oligonucleotides within the adapter, Different fluorescent dyes can be introduced as labels into synthetic oligonucleotides as for example commercially available from Eurofins [http://www.euroflnsdna.com/products-services/oligonucleotides/modifications/dna-modifications/fluorescent-dyes.html], or for example by the use of a cross linking group at the 3′ end of the oligonucleotide. Preferable fluorescent dyes for practicing the invention have been published by Ikeda S. and Okamoto A. Chem. Asian J. 3, 958-968 (2008). Alternatively fluorescent dyes can be introduced at the 3′ end of an oligonucleotide in a reaction mediated by a terminal transferase as described in the forgoing. Preferably the oligonucleotide ligated to the 3′ end of the cDNA on the solid support carries the label. Thus the label can be used to mark the location of cDNA molecules on the solid support to which a priming site had been added. Such a label can be an important quality control to understand the yield of the 3′end ligation step and may be used in the analysis of sequences obtained from the same location on a solid support.

Alternatively, a priming site at the 3′ end of cDNA can be introduced by addition of homopolymers by means of Terminal deoxynucleotidyl transferase. The Terminal deoxynucleotidyl transferase adds nucleotides to the 3′ end of DNA in a template free reaction, where commonly only one nucleotide is offered in the reaction mix. Hence the enzyme will extend the cDNA molecules by adding homopolymers to the 3′ end. Preferably oligo-dG stretches are added in the reaction as for example describe in Okayama H. and Berg P., Mol Cell Biol 2, 161-170 (1982). Terminal deoxynucleotidyl transferase can be purchased from different providers including New England BioLabs and Fermentas. Most preferably, the priming site is introduced by ligation of a double-stranded adaptor to the 3′ end of cDNA molecules. In a second reaction mediated by a terminal transferase, the enzyme may be used to add a label to the 3′ end of the homopolymer synthesized in the first reaction. Conditions for labeling for example a polyA-tailed DNA in a terminal transferase reaction with cyanine 3-ddTTP can be found in the supplementary information of Harris T D et al., Science 320, 106-109 (2008).

In an alternative embodiment, RNA molecules modified at their 5′ end or having a known sequence at their S′ end may not require the addition of a priming site to the 3′ end of the cDNA, but known sequences can be used to prime a primer extension reaction. Such priming site may be derived from an RNA molecule to which an oligonucleotide of known sequence had been added to the 5′ end. In a different embodiment, priming sequences are selected by computational means as for example deposited in public databases. In another example, sequences specific for 5′ ends of mouse and human mRNA have been reported in the literature [Carninci P. et al., Nat. Genetics 38, 626-35 (2006) and Carninci P. Et al., Science 309, 1559-63 (2005)]. Additional 5′ end specific sequences from mRNA have been for example obtained at a very large scale from 5′ EST sequencing projects as well as from CAGE, 5′ SAGE and PET sequencing studies. Hence, 5′ end specific sequences from known transcripts can be used to design sequencing primers to obtain sequence information from the true 5′ ends of mRNAs. Such sequencing primers allow for specific priming at 5′ ends of mRNAs. In a different embodiment, primers are designed from any sequence derived from RNA transcripts or a genome available for primer design to enable sequencing of defined regions within target molecules attached to a solid support. In a specific embodiment, the defined sequences are splice sites to identify splicing patterns or alternative use of promoters. In another specific embodiment, the defined sequences resemble sequences found in other RNA molecules such as short RNAs. In this embodiment, sequencing primers have identical, nearly identical, complementary, or nearly complementary sequence to the entire or part of the sequence of an RNA molecule. Such sequencing primers can then drive sequencing reactions to identify flanking sequences within parental RNA molecules processed during the maturation of short RNA. Moreover, such sequencing primers may be used to identify target RNAs participating in a direct interaction between two RNA molecules. In just another embodiment, the sequencing primers are having a random sequence, and the sequencing reactions lead to results similar to a shotgun sequencing reaction. Since the cDNA template is attached to a solid support, it is within the scope of the invention to perform more than 1 sequencing reaction on an immobilized cDNA template including, but not limited to, the forgoing applications. It is within the scope of the invention to perform multiple sequencing reactions using a cDNA template Immobilized on a solid support. Therefore it is within the scope of the invention to perform multiple cycles comprising a hybridization reaction of a sequencing primer to the template on the solid support, conducting a sequence reaction to obtain sequence information from a regions contained in the template on the solid support, to remove the extended sequencing primer, and to enter into a new cycle.

The priming site can be used to prime synthesis of a second DNA to prepare a double-stranded DNA molecule on the solid support (FIG. 13). Different approaches for the synthesis of a second DNA strand by means of a DNA polymerase can be found for example in Sambrook J, and Russuell D. W., Molecular Cloning, A Laboratory Manual, Cold Spring Harbor Laboratory Press, New York, 2001. DNA polymerases suitable for DNA synthesis include but are not limited to the Klenow fragment of DNA polymerase I, T4 and T7 DNA polymerases, DNA polymerase I, Taq polymerase, Tfl DNA polymerase, Tth DNA polymerase, Tli DNA polymerase, or any other DNA polymerase known in the field or otherwise commercially available on the market. In one embodiment, the synthesis of a second strand will extend the primer up to the 5′ end of the cDNA molecule on the solid support. In a different embodiment, the synthesis of a second strand will be terminated before reaching the 5′ end of the cDNA molecule on the solid support. In just a different embodiment, the synthesis of a second strand will be prepare random DNA fragments having complementary sequence to parts of the cDNA attached to the solid support. In just a different embodiment, the second strand is made of RNA and the synthesis is done by a DNA dependent RNA polymerase. In this embodiment, the DNA dependent RNA polymerase can be used to amplify the template on the solid support. In one such reaction, the DNA dependent RNA polymerase will synthesize more than one RNA copy of the template. In another reaction, RNA copies prepared by means of the DNA dependent RNA polymerase carry a priming site to drive a reverse transcription reaction. In a reaction combining RNA and DNA synthesis, an exponential amplification of an RNA and/or DNA template is possible. Such amplification products may be used to obtain sequence information by means of the present invention, may be used to perform a shotgun sequencing experiment, may be used to clone specific transcripts, or may be used to clone a cDNA library. In just a different embodiment, the DNA dependent RNA polymerase may derive a sequencing reaction.

It is within the scope of the inventions to use different reaction steps to prepare fragments for shotgun sequencing, and to mix the resulting fragments for sequencing. For example, random fragments can be obtained prior to applying the invention, during first strand synthesis, second strand synthesis, or any DNA or RNA products prepared during the conduct of the invention.

Preferably, the priming site introduced at the 3′ end of the cDNA is used to drive a sequencing reaction to obtain sequence information from the 5′ end of the original RNA template. Different protocols for the sequencing of DNA by means of a DNA polymerase have been published as for example reviewed in Metzker M. L. Genome Res. 15, 1767-1776 (2005), Kling J., Nature Biotechnology 23, 1333-1335 (2005), Shendure J. et al., Nature Review Genetics 5, 335-344 (2004), Mardis E. R. Trends in Genetics 24, 133-141 (2008), and von Bubnoff A. Cell 132, 721-723 (2008). To practice the invention, DNA sequencing is preferably conducted by a sequencing-by-synthesis method that detects the incorporation of each nucleotide. The incorporation reaction may detect for different nucleotide having 4 different dyes at the same time, or 4 different nucleotides are tested for incorporation in separate reactions. Different methods for sequencing-by-synthesis for DNA sequencing have been described in the literature and are commercially available for example from 454, now ROCHE, Illumina, or Helicos BioSciences. Such approaches make use of labeled nucleotides to monitor the incorporation of such a labeled nucleotide in an extension reaction (Illumina and Helicos), or otherwise monitor reaction products derived from an extension reaction as for example the pyrophosphate molecule cleaved of from the incorporated nucleotide (454 and Pyrosequencing). In this reaction, the sequence of a number of nucleotides is determined, where the number of nucleotides may be smaller than number of nucleotides in the DNA template. In an alternative embodiment, the invention may make use of other sequencing approaches such as sequencing by hybridization or ligation.

The sequence information from the 5′ end of RNA may be extended by repeating cycles at a number of time, each determining a short stretch of sequence (FIG. 15). Each cycle comprises the following reaction steps:
An adapter with a priming site is added to the 3′ end of a DNA molecule attached to a solid support. This priming site has, adjacent to the 3′ end of the DNA molecule, a recognition site for a restriction endonuclease that cuts outside of its binding site.
A sequencing primer hybridizes to the primer site and drives a sequencing reaction. This sequencing reaction starts at the nucleotide adjacent to the 3′ end of the DNA molecule attached to a solid support. The number of nucleotides sequenced during the sequencing step is related to the number of nucleotides removed in the digestion step.
The restriction endonuclease that cuts outside of its binding site is used to remove a DNA fragment from the end of the DNA molecule attached to a solid support
A new adapter with a priming site is added to the end of a DNA molecule attached to a solid support opened by the digestion step using the restriction endonuclease that cuts outside of its binding site. The priming site has adjacent to the 3′ end of the DNA molecule a recognition site for a restriction endonuclease that cuts outside of its binding site. The adapter with a priming site may be the same as the one used in the initial step.
A sequencing primer hybridizes to the primer site and drives a second sequencing reaction, thus starting a new round to obtain extended sequence information from the S′ end of RNA.
The reaction chain can be repeated in a cycling process to obtain extended sequence information from a DNA molecule attached to a solid support.

In a different embodiment, the reaction cycle is preformed in solution. The length of the new sequence information obtained by each round is determined by the restriction endonuclease. Since the restriction endonuclease that cuts outside of its binding site can only cut double-stranded DNA, the enzyme cuts off only DNA regions for which the a second cDNA strand had been synthesized during the sequencing reaction, and not within single-stranded cDNA. Hence the process can be controlled in such a way that no internal digestion of DNA molecules on the solid support occurs. A preferable enzyme that cleaves outside of its recognition sequence is the Class IIs restriction enzyme MmeI cleaving 20/18 base pairs apart from it recognition site. A more preferable enzyme that cleaves outside of its recognition sequence is the Class III restriction enzyme EcoP15I cleaving 25/27 base pairs apart from it recognition site. In case EcoP15I is used in the digestions step, the adapter may have 2 recognition sites for binding by EcoP15I. Both enzymes are commercially available from New England BioLabs. If the restriction endonuclease is MmeI, preferably 25 nucleotides are sequenced, If the restriction endonuclease is EcoP15I, preferably 30 nucleotides are sequenced. The sequencing reaction should extend the DNA strand to provide a double-stranded DNA long enough for digestion by the restriction endonuclease. In addition, re-sequencing of 3, 4, 5 or 6 nucleotides may be helpful to assemble longer sequences from individual reads. Similarly, any other restriction endonuclease cutting outside of its binding site may be used within such cycle to obtaining sequence information.

In the forgoing, conditions for different reaction steps have been described, However, the invention allows for different combinations of those reaction steps to obtain sequence information from the 5′ end, the 3′ end, or the 5′ end and 3′ end of an RNA molecule. In a different embodiment, the invention may be used to obtain random sequence for an RNA molecule.

A preferred mode of the invention comprises the following reaction steps to obtain sequence information from the 5′ ends of RNA molecules within a sample (Table 3):
1. RNA molecules in an RNA sample are modified at their 3′ end so as to introduce a functional group in a polyadenylation reaction.
2. RNA molecules having a polyA tail at the 3′ end are extended by adding a polyC tail to their 3′ end.
3. Diol groups of RNA molecules within the RNA samples are labeled introducing a fluorescent dye.
4, Labeled RNA molecules are bound to a solid support by binding of the functional group to a capturing oligonucleotide on the solid support. Since the functional group is comprised of a polyA tail, the capturing oligonucleotide is made of an oligo-dT oligonucleotide.
5. Labeled RNA molecules are detected on the solid support to determine loading of the solid support and to determine the percentage of capped mRNAs within the sample.
6. The capturing oligonucleotide primes a reverse transcription reaction to synthesize cDNA molecules attached to the solid support and having a sequence complementary to the RNA template bound to the capturing oligonucleotide.
7. RNase I digestion removes labels from 3′ ends of RNA molecules, and from Cap structure of DNA-mRNA hybrids that have not been extended to reach the 5′ end of mRNA templates.
8. Full-length cDNAs for capped mRNAs are detected on the solid support to determine the percentage of capped mRNAs or full-length cDNAs within the sample.
9. RNA templates are removed.
10. A double-stranded adapter with a priming site is ligated to the 3′ end of the single stranded cDNA on the solid support.
11. The priming site within the adapter is used to drive a sequence reaction to obtain sequence information from the 5′ end of RNA molecules within the sample.

TABLE 3
Principle process flow (option 1)

| Step No | Description | Comment |
|---|---|---|
| 1 | Polyadenylation of RNA or ligation | All RNA have common sequence to bind |
| | of oligonucleotide to 3′ end | to solid support |
| 2 | Optional 2nd polyadenylation | To assure free 3′-end after binding to |
| | reaction to add C tail | solid support |
| 3 | Labeling of diol groups in RNA | Mark location on solid support and need |
| | | for labeling 5′ end of full-length RNAs |
| 4 | Binding to sold support in | Immobilization of template for further |
| | hybridization reaction | manipulations |
| 5 | Label scanning | Identifying positions of RNAs on solid |
| | | support |
| 6 | Fill-in reaction to reach 5′ end of | Required to sequence 5′ end of RNA |
| | RNA | |
| 7 | Label scanning | Identifying positions of full-length |
| | | mRNAs on solid support |
| 8 | Remove RNA from cDNA | Leaves cDNA template on solid support |
| 9 | Add linker or poly G tail to 3′ end of | Required for priming site |
| | cDNA | |
| 10 | DNA sequencing step | Sequencing 5′ end of RNA |

Another preferred mode of the invention comprises the following reaction steps to obtain sequence information from the 3′ ends of RNA molecules within a sample (Table 4):
1. RNA molecules in an RNA sample are modified at their 3′ end so as to introduce a functional group in a polyadenylation reaction.
2. RNA molecules having a polyA tail at the 3′ end can optionally be extended by adding a polyC tail to their 3′ end.
3. Diol groups of RNA molecules within the RNA samples are labeled introducing a fluorescent dye.
4. Labeled RNA molecules are bound to a solid support by binding of the functional group to a capturing oligonucleotide on the solid support. Since the functional group is comprised of a polyA tail, the capturing oligonucleotide is made of an oligo-dT oligonucleotide.
5. Labeled RNA molecules are detected on the solid support to determine loading of the solid support and to determine the percentage of capped mRNAs within the sample.
6. The capturing oligonucleotide primes a reverse transcription reaction to synthesize cDNA molecules attached to the solid support and having a sequence complementary to the polyA tail of the RNA template bound to the capturing oligonucleotide.
7. The extended capturing oligonucleotide primes a reverse transcription reaction to obtain sequence information from the 3′ end of RNA molecules within the sample.

TABLE 4
Principle process flow (option 2)

| Step No | Description | Comment |
|---|---|---|
| 1 | Polyadenylation of RNA or | All RNA have common sequence to bind to |
| | Ligation of oligonucleotide to 3′ end | solid support |
| 2 | Optional 2nd polyadenylation reaction | To assure free 3′-end after binding to solid |
| | to add C tail | support |
| 3 | Labeling of diol groups in RNA | Mark location on solid support and need for |
| | | labeling 5′ end of full-length RNAs |
| 4 | Binding to sold support in | Immobilization of template for further |
| | hybridization reaction | manipulations |
| 5 | Label scanning | Identifying positions of RNAs on solid |
| | | support |
| 6 | Fill-in reaction | Required to reach starting point of |
| | | sequencing reaction |
| 7 | RNA sequencing step | Sequencing 3′ ends of RNA |

In just another preferred mode of the invention comprises the following reaction steps to obtain sequence information from the 3′ ends and 5′ ends of RNA molecules within a sample (Table 5):
1. RNA molecules in an RNA sample are modified at their 3′ end so as to introduce a functional group in a polyadenylation reaction.
2. RNA molecules having a polyA tail at the 3′ end are extended by adding a polyC tail to their 3′ end.
3. Diol groups of RNA molecules within the RNA samples are labeled introducing a fluorescent dye.
4. Labeled RNA molecules are bound to a solid support by binding of the functional group to a capturing oligonucleotide on the solid support. Since the functional group is comprised of a polyA tail, the capturing oligonucleotide is made of an oligo-dT oligonucleotide.
5. Labeled RNA molecules are detected on the solid support to determine loading of the solid support and to determine the percentage of capped mRNAs within the sample.
6. The capturing oligonucleotide primes a reverse transcription reaction to synthesize cDNA molecules attached to the solid support and having a sequence complementary to the polyA tail within the RNA template bound to the capturing oligonucleotide.
7. The extended capturing oligonucleotide primes a reverse transcription reaction to obtain sequence information from the 3′ end of RNA molecules within the sample.
8. The extended capturing oligonucleotide primes a reverse transcription reaction to synthesize cDNA molecules attached to the solid support and having a sequence complementary to the RNA template bound to the capturing oligonucleotide.
9. RNase I digestion removes labels from the 3′ ends of RNA molecules and from the Cap structures of cDNA-mRNA hybrids that have not been extended to reach the 5′ end of mRNA templates.
10. Full-length cDNAs for capped mRNAs are detected on the solid support to determine the percentage of capped mRNAs or full-length cDNAs within the sample.
11. RNA templates are removed.
12. A double-stranded adapter with a priming site is ligated to the 3′ end of the single-stranded cDNA on the solid support.
13. The priming site within the adapter is used to drive a sequence reaction to obtain sequence information from the 5′ end of RNA molecules within the sample.

TABLE 5
Principle process flow (option 3)

| Step | | |
|---|---|---|
| No | Description | Comment |
| 1 | Polyadenylation of RNA or | All RNA have common sequence to bind to |
| | Ligation of oligonucleotide to 3′ | solid support |
| | end | |
| 2 | Optional 2nd polyadenylation | To assure free 3′-end after binding to solid |
| | reaction to add C tail | support |
| 3 | Labeling of diol groups in RNA | Mark location on solid support and need for |
| | | labeling 5′ end of full-length RNAs |
| 4 | Binding to sold support in | Immobilization of template for further |
| | hybridization reaction | manipulations |
| 5 | Label scanning | Identifying positions of RNAs on solid |
| | | support |
| 6 | Fill-in reaction | Required to reach starting point of sequencing |
| | | reaction |
| 7 | RNA sequencing step | Sequencing 3′ ends of RNA |
| 8 | Fill-in reaction to reach 5′ end of | Required to sequence 5′ end of RNA |
| | RNA | |
| 9 | Label scanning | Identifying positions of full-length mRNAs on |
| | | solid support |
| 10 | Remove RNA from cDNA | Leaves cDNA template on solid support |
| 11 | Add linker or poly G tail to 3′ end | Required for priming site |
| | of cDNA | |
| 12 | DNA sequencing step | Sequencing 5′ end of RNA |

In a special embodiment, the invention targets at the analysis of mRNA molecules obtained from a single-cell. Sequence information from regions within mRNA molecules can be obtained in just another preferred mode of the invention comprising the following reaction steps to obtain sequence information from the 3′ ends and 5′ ends of mRNA molecules within a sample (Table 6):
1. Total RNA contained in the sample is incubated with a solid support having capturing oligonucleotides having an oligo-dT sequence.
2. Polyadenylated mRNA molecules within the sample hybridize to solid support whereas nonpolyadenylated RNA molecules do not bind to the solid support.
3. RNA molecules not binding the solid support are washed away.
4. Diol groups within mRNA molecules on the solid support are labeled by introducing a fluorescent dye.
5. Labeled mRNA molecules are detected on the solid support to determine loading of the solid support and to determine the percentage of capped mRNAs within the sample.
6. The capturing oligonucleotide primes a reverse transcription reaction to synthesize cDNA molecules attached to the solid support and having a sequence complementary to the polyA tail within the mRNA template bound to the capturing oligonucleotide.
7. The extended capturing oligonucleotide primes a reverse transcription reaction to obtain sequence information from the 3′ end of mRNA molecules on the solid support.
8. The extended capturing oligonucleotide primes a reverse transcription reaction to synthesize cDNA molecules attached to the solid support and having a sequence complementary to the mRNA template bound to the capturing oligonucleotide.
9. RNase I digestion removes labels from the 3′ ends of mRNA molecules and from the Cap structure of cDNA-mRNA hybrids that have not been extended to reach the 5′ end of mRNA templates.
10. Full-length cDNAs for capped mRNAs are detected on the solid support to determine the percentage of capped mRNAs or full-length cDNAs within the sample,
11. RNA templates are removed.
12. A double-stranded adapter with a priming site is ligated to the 3′ end of the single-stranded cDNA on the solid support.
13, The priming site within the adapter is used to drive a sequence reaction to obtain sequence information from the 5′ end of mRNA molecules within the sample.

These reaction steps may be modified to obtain only sequence information from the 3′ end of mRNA, or only from the 5′ end of mRNA.

TABLE 6
Principle process flow (option 4)

| Step No | Description | Comment |
|---|---|---|
| 1 | Total RNA incubated with solid | Selective binding of polyadenylated |
| | support having oligo-dT capturing | mRNA |
| | oligonucleotide | |
| 2 | Removal of nonpolyadenylated RNA | Enrichment of mRNA on solid support |
| 3 | Labeling of diol groups in mRNA | Mark location on solid support and |
| | | need for labeling 5′ end of full-length |
| | | mRNAs |
| 4 | Label scanning | Identifying positions of mRNAs on |
| | | solid support |
| 5 | Fill-in reaction | Required to reach starting point of |
| | | sequencing reaction |
| 6 | RNA sequencing step | Sequencing 3′ ends of mRNA |
| 7 | Fill-in reaction to reach 5′ end of | Required to sequence 5′ end of mRNA |
| | mRNA | |
| 8 | Label scanning | Identifying positions of full-length |
| | | mRNAs on solid support |
| 9 | Remove mRNA from cDNA | Leaves cDNA template on solid support |
| 10 | Add linker or poly G tail to 3′ end of | Required for priming site |
| | cDNA | |
| 11 | DNA sequencing step | Sequencing 5′ end of mRNA |

The present invention encompasses different methods for obtaining additional sequence information from different regions of RNA molecules. It is within the scope of the present invention to perform more than one sequence reaction on template RNA or template DNA attached to the solid support. In one embodiment, specific sequencing primers are used to drive one or more sequencing reactions using a reverse transcriptase and an RNA template. In another embodiment, specific sequencing primers are used to drive one or more sequencing reactions using a DNA polymerase and a DNA template. In just another embodiment, specific promoter regions are used to drive one or more sequencing reactions using a DNA-dependent RNA polymerase and a DNA template. Multiple sequencing reactions may be used to obtain sequence information, consecutively or randomly, or to obtain sequence information from defined regions within RNA molecules. Moreover, it is within the scope of the invention to obtain sequence information from immobilized templates at different times and in separate sequencing experiments. Hence, localization patterns of labeled molecules on the solid support can be used to identify specific solid supports and molecules attached to it for use in extended or otherwise repetitive experiments. The location of molecules on the solid support may be identified by use of labels added to the 3′ end of RNA or the 3′ end of a cDNA bound to the solid support. Multiple sequencing experiments using the same solid support may be performed at different time points, as needed to design new sequencing primers based on sequencing information obtained in a previous sequencing run.

In just a different embodiment, cDNA molecules attached to the solid support are used as templates to prepare a cDNA or cDNA library. A person skilled in the art knows different approaches to cDNA synthesis and release of cDNAs from a solid support to prepare an individual cDNA or to prepare a cDNA library. In a preferable embodiment, solid supports are maintained after conducting the analysis of the molecules in one or more sequencing reactions. Sequences derived from the analysis of the molecules on the solid support can then be used to design specific primer sets for cloning of dedicated cDNAs. Such a cloning step can make use of an amplification reaction including but not limited to, a PCR reaction. Cloning of PCR products may make use of special features introduced by the PCR primers, or otherwise can make use of special features introduced by a functional group or an adapter used in the course of the invention. Hence, the invention provides a method for analyzing and cloning RNA molecules obtained from a sample.

The invention provides a method for obtaining sequence information from RNA molecules within a sample. Preferably such a sample comprises all RNA molecules including different RNA species as contained in a cell, tissue, or organism. Hence, the invention targets at the parallel analysis of all RNA molecules within the sample regardless of which category of RNA they may belong to. The computational analysis of sequences obtained by means of the invention should therefore include steps that make in particular use of the ability to analyze all RNA species at the same time and to look for direct interactions between RNA molecules. As outlined in FIG. 18 a computational analysis of the sequence information can comprise the following steps:
1. High quality sequences are extracted from row sequencing reads.
2. High quality reads are linked to the location information obtained by the readout of the labels.
3. Use labeling information to mark reads obtained from mRNA molecules and full-length cDNAs.
4. Sequences obtained from the same location are grouped.
5. Sequences are assembled into longer sequences if more than one sequence had been obtained from the 5′ end of RNA molecules in consecutive sequencing reactions.
6. Sequences within each group or from assembled sequences are mapped to a suitable genome.
7. Mapping positions in the genome identify the transcripts and provide annotation information on the transcribed regions.
8. Sequences mapping to the same location in the genome are counted to obtain digital expression profiles.
9. Mapping positions can identify promoter regions as identified by mapping sequences obtained from 5′ ends of mRNA. Sequences obtained from 5′ ends of non mRNA molecules may identify promoter regions, provided such RNA molecules have not been processed in the cell to generate mature RNA species.
10. Motive searches around the mapping positions can identify RNA molecules that have been processed in the cell to generate mature RNA species.
11. Mapping positions in the gnome may identify trans spliced transcripts where reads from the 5′ end and 3′ end of the same RNA molecule map to different chromosomes.
12. Sequence information from the transcribed regions is retrieved from genome sequences.
13. Sequence information from transcribed regions is used in computational alignments to identify interactions between RNA molecules having complementary or in part complementary sequence.
14. Sequencing reads may be analyzed for the presence of an Identifier Sequence and/or the presence of an internal control. Sequences obtained from Identifier Sequences may be counted to obtain information on the contribution of each sample within a pooled library. Sequences obtained from internal controls may be used to calculate the recovery rates of an experiment or otherwise to estimate the bias of an experiment.

Sequencing reads and any sequence derived thereof can be analyzed for their identity by mapping to genomic regions or otherwise by putting them into relationship with known sequences in databases. Most commonly used databases include, but are not limited to:

NCBI (http://www.ncbi.nlm.nih.gov/Database/index.html),
EMBL-EBI (http://www.ebi.ac.uk/Databases/index.html), or
DNA Data Bank of Japan (http://www.ddbj.nig.ac.jp/).

Those and other databases available in public domain provide access to genome sequences. For example, genome sequence databases are provided by the NCBI and can be found under:

http://www.ncbi.nlm.nih.gov/genomes/lltp.cgi or
http://www.ncbi.nlm.nih.gov/sites/entrez?db=genomeprj.

Individual sequences can be analyzed for their identity by standard software solutions to perform sequence alignments, including, but not limited to, NCBI BLAST (http://www.ncbi.nlm.nih.gov/BLAST/) or FASTA, available in the Genetics Computer Group (GCG) package from Accelrys Inc. (http://www.accelrys.com/). A person skilled in the art knows different alignment tools or software solutions along with their appropriate settings for the alignment of sequencing reads to genomic sequences or for alignment of sequences against each other to identify interacting RNA molecules. Such software solutions further allow identification of unique or non-redundant sequences mapping to defined locations in the genome. All such non-redundant sequences can then be individually counted and further analyzed for the contribution of each non-redundant transcript to the total number of all transcripts obtained from the same sample. The contribution of an individual transcript to the total number of all transcripts within the sample enables the quantification of the transcripts within a plurality of RNA molecules contained in the sample. The results obtained in such a way on individual samples can be further compared to similar data obtained from other samples. Hence the invention provides a method for comparing expression profiles obtained from different samples in expression profiling studies. Thus, the invention allows for the expression profiling of individual transcripts within one or more samples, gene discovery by de novo sequencing, and the establishment of a reference database. Sequence information obtained by means of the invention can further be used retrieve sequence information from transcribed regions within genomes. Preferably, sequence information of transcribed regions is identified by obtaining sequence information from both ends of an RNA molecule that can be mapped to genomic sequences. Sequences from expressed regions provide important resources to identify RNA-RNA interactions and parental transcripts giving raise to short RNAs during a maturation process. Genomic sequence information is required in particular for identifying parental transcripts for short RNAs derived from introns, which are no longer found in spliced mRNAs.

In a different embodiment, genomic sequences are analyzed to identify sense-antisense pairs as commonly found in transcribed regions [Katayama S. et al., Science 309, 1564-6 (2005)]. In just a different embodiment, genomic sequences are use to retrieve annotation information including, but not limited to, information on transcribed regions, open chromatin, functional elements in the genome, point mutations or other genetic alterations, and/or genome modifications like for instance methylation patterns.

In just a different embodiment, the present invention relates to the design and preparation of hybridization probes and sequencing primers based on sequence information obtained by means of the invention. In a different embodiment, hybridization probes as derived from sequencing information are used in in situ hybridization experiments. Such experiments include but are not limited to the use microarrays. In a preferable embodiment the microarray is a tiling array, where oligonucleotides or DNA fragments on the array cover in part or entire genomic regions. In just a different embodiment, probes or cDNAs prepared by means of the invention are annotated by hybridization to a microarray or set of microarrays, where such a microarray or microarrays preferably comprises genomic regions. A solid support having cDNA molecules on its surface as derived from the RNA contained within a sample may be used as a microarray in a hybridization reaction. Such microarray may be used in a sequencing by hybridization reaction, to detect RNA molecules, or to study alternative expression of genes within more than one samples. The microarray can be annotated by the sequence information obtained by means of the present invention.

The present invention provides new methods for obtaining information to describe a biological system and using such genetic information. Hence the present invention enables the design and performance of analytical assays for studies in life science and diagnosis. In particular, the present invention enables new methods for studying expression profiles, activities of promoters, and regulatory networks.

The present invention or any parts thereof can be used for the production of a kit containing among other components the reagents, an internal control, nucleic acid molecules, and/or enzymes for the manipulation of RNA, the preparation of cDNA, a microarray, a solid support and to perform one or more sequencing reactions. In one embodiment a kit provides the reagents needed to sequence RNA. In a different embodiment a kit provides the reagents to sequence DNA. In just a different embodiment a kit provides the reagents to sequence RNA and DNA. In a preferable embodiment a kit provided the reagents to prepare a template for single molecule detection. In another preferable embodiment a kit provides the reagents for a research purpose. In an even more preferable embodiment a kit provides the reagents for a diagnostic assay.

### EXAMPLES

The present invention will be further explained by way of the following examples. These examples provide typical reaction conditions to practice individual steps according to some embodiments of the invention, but the present invention is not limited to the conditions given in the examples. All names and abbreviations as used herein shall have the meaning as commonly used in the field and known to a person skilled in the art.

Standard reagents for experiments in molecular biology including enzymes and nucleotides can be commercially obtained from different suppliers including but not limited to, FERMENTAS (Vilnius, Lithuania), New England Biolabs (Beverly, USA), Promega (Madison, USA), Takara (Tokyo, Japan), Roche (Mannheim, Germany), or GE Biosciences (Cardiff, United Kingdom).

Special precautions have to be taken for working with RNA to avoid RNA degradation. For further details on how to work with RNA refer to Sambrook J. and Russuell D. W., Molecular Cloning, A Laboratory Manual, Cold Spring Harbor Laboratory Press, New York, 2001, or other text books.

### Example 1

### Isolation of RNA

First, total RNA samples are prepared using commercial reagents and standard methods known to a person skilled in the art of molecular biology, as given in more detail, for example, in Sambrook J. and Russuell D. W., Molecular Cloning, A Laboratory Manual, Cold Spring Harbor Laboratory Press, New York, 2001. Furthermore, Carninci P. et al. described in Biotechniques 33, 306-309 (2002) a method to obtain cytoplasmic RNA fractions. It is within the scope of the invention to prepare total RNA after cell fractionation, namely from the nucleus and cytoplasm of cells.

The quality of RNA samples can be analyzed by the ratios of the OD readings at 230, 260 and 280 nm to monitor RNA purity. Removal of polysaccharides is considered successful when the 230/260 ratio is lower than 0.5, and an effective removal of proteins is achieved when the 260/280 ratio is higher than 1.8 or around 2.0. The RNA samples can further be analyzed by electrophoresis in an agarose gel or the use of an Agilent 2100 bioanalyzer to prove the ratio between the 28S and 18S rRNA in total RNA preparations (note rRNA size may change for preparation of total RNA from other species than mammalians), and to check the integrity of the RNA samples.

### Example 2

### Pyrophosphatase Reaction to Remove Cap Structure

The total RNA sample is treated with a pyrophosphatase to remove the Cap structure from the 5′ end of mRNA. In a typical reaction 3 μg of total RNA are incubated at 65° C. for 5 min in a total volume of 42.9 μl water to destroy secondary structures. The RNA is afterwards chilled on ice until setting up the reaction by adding:

| | |
|---|---|
| RNA (3 μg) | 42.9 μl |
| 10x TAP buffer | 5 μl |
| Cloned RNase Inhibitor (40 U/μl (Takara) | 2 μl |
| Tobacco acid pyrophosphatase (150 U/μl) | 0.1 μl |
| Total volume | 50 μl |

After incubation at 37° C. for 1 h, the reaction mixture is extracted with 50 μl phenol/chloroform, followed by 50 μl chloroform only. For further purification the RNA is precipitated with isopropanol using glycogen as a carrier:

| | |
|---|---|
| 1 μg/μl Glycogen | 3 μl |
| 5M NaCl | 5 μl |
| Isopropanol | 100 μl |

After incubation at −20° C. for at least 30 min or overnight, the precipitate is collected by centrifugation at 15,000 rpm and 4° C. for 30 min. The pellet is washed first with 800 μl of 80% ethanol and second time 100 μl of 80% ethanol before the pellet is finally dissolved in 10 μl 0.1×TE buffer.

### Example 3

### Ligation of an Oligonucleotide to the 3′ End of RNA

Ligation of an RNA oligonucleotide to the 3′ end of RNA can be performed by means of an RNA ligase. Features of the RNA oligonucleotide are further described in the description of the invention.

To conduct the ligation reaction, 1 μg of total RNA is mixed with 100 pmol of RNA oligonucleotide, and water is added up to a final volume of 15.34 μl. The mixture is incubated at 65° C. for 5 min and placed on ice. For the ligation reaction the following reagents are added:

| | | |
|---|---|---|
| RNA sample and oligonucleotide | 15.34 | μl |
| 50% PEG 6000 | 25 | μl (final 25%) |
| 10x T4 RNA buffer | 5 | μl |
| 0.1% BSA (0.006%) | 3 | μl |
| T4 RNA ligase (Takara) | 1.7 | μl (50 Units) |
| Total volume | 50 | μl |

The reaction mixture is incubated overnight at 15° C. before terminating the reaction by destruction of the RNA ligase by Proteinase K treatment:

| | |
|---|---|
| 0.1x TE buffer | 50 μl |
| 0.5M EDTA | 2 μl |
| 10% SDS | 2 μl |
| Proteinase K | 2 μl |

After incubation at 37° C. for 15 min, the RNA is purified by ethanol precipitation:

| | |
|---|---|
| Sample | 100 μl |
| 5M NaCl | 5 μl |
| Glycogen | 2 μl |
| 100% Ethanol | 250 μl |

The RNA is precipitated at −20° C. for at least 30 min or overnight, before collecting the RNA by centrifugation at 15000 rpm and 4° C. for 30 min. The pellet should be washed first with 800 μl of 80% ethanol, and second time with 100 μl of 80% ethanol. The purified RNA can be dissolved in 20 μl water.

### Example 4

### Polyadenylation Reaction

Alternatively, RNA can be modified by the addition of a poly A tail using a Poly(A) polymerase. In a standard reaction 10 μg of the total RNA is used:

| RNA sample (10 μg) | x | μl |
|---|---|---|
| 10 times Poly (A) polymerase buffer containing | 10 | μl |
| 1 mM ATP | | |
| E. coli Poly (A) Polymerase (NEB, #M0276S) | y | μl (5 Units) |
| Water up to a final volume of | 100 | μl |

After incubation at 37° C. for 10 min, 1 μl of 0.5 M EDTA (pH 8.0) is added to stop the reaction, followed by extraction with an equal volume of phenol/chloroform and chloroform. The RNA is purified by ethanol precipitation as described in other Examples herein by adding 2.5-fold volume of ethanol. The precipitated RNA is harvested by centrifugation at 15000 rpm and 4° C. for 15 min. Purified RNA is dissolve in water prior to further use.

The reaction can be modified to remove diol groups from the 3′ end of RNA. In such a case, total RNA, RNA obtained from a ligation or a polyadenylation reaction is mixed with 5 μl of 10 times Poly(A) polymerase buffer without ATP, 2.5 units of E. coli Poly(A) polymerase and 1 μl of 10 mM 3′-deoxyATP, in a total volume of 50 μl. After incubation at 37° C. for 10 min, 1 μl of 0.5 M EDTA (pH 8.0) is added to stop the reaction, and the RNA is purified as described above.

### Example 5

### Oxidation of Diol Groups within RNA Molecules

The diol groups in RNA molecules can be oxidized under the following conditions to create a reactive group for introducing a label 50 g of RNA is used:

| RNA (50 μg) | x | μl |
|---|---|---|
| 1M NaOAc (pH 4.5) | 3.3 | μl |
| 250 mM NaIO₄ | 2 | μl |
| Add water to a final volume of | 51.3 | μl |

The reaction tube has to be wrapped with aluminum foil to avoid any exposure to light, and the reaction is conducted on ice for 45 min. The reaction is terminated by adding 1 μl of 80% glycerol. After mixing the sample quickly with the glycerol, the RNA is precipitated with isopropanol:

| | |
|---|---|
| 10% SDS | 0.5 μl |
| 5M NaCl | 11 μl |
| Isopropanol | 61 μl |

After incubation at −20° C. for at least 30 min, the RNA is collected by centrifugation at 15000 rpm and 4° C. for 30 min. The RNA pellet is washed first with 800 μl of 80% ethanol and second time with 100 μl of 80% ethanol, before dissolving the pellet in 50 μl water.

### Example 6

### Introduction of Label

A fluorescence label can be added to the oxidized RNA from the reaction products of Example 5. In the reaction given here, Cy5 Hydrazaide is used as an example:

| | | |
|---|---|---|
| Oxidized RNA | 50 | μl |
| 1M NaOAc (pH6.1) | 5 | μl |
| 10% SDS | 5 | μl |
| 10 mM Cy5 Hydrazaide | 150 | μl |

The reaction components are mixed and incubated at room temperature for 10-12 h, before the reaction is terminated and the RNA is purified by ethanol precipitation:

| | | |
|---|---|---|
| 1M NaOAc (pH6.1) | 75 | μl |
| 5M NaCl | 5 | μl |
| 100% ethanol | 750 | μl |

After incubation at −20° C. for at least 30 min, the RNA is collected by centrifugation at 15000 rpm and 4° C. for 30 min. The RNA pellet is washed first with 800 μl of 80% ethanol and second time with 100 μl of 80% ethanol, before dissolving the pellet in 50 μl of 0.1×TE buffer.

### Example 7

### Hybridization Reaction of RNA to Oligonucleotides Immobilized on a Solid Support

In a hybridization reaction RNA having a polyA tail at the 3′ end is bound to a solid support having oligo-dT oligonucleotides on its surface. Solid supports with immobilized oligo-dT oligonucleotides are commercially available and are commonly used to purify mRNA fractions from total RNA. A preferable solid support is modified Dynabeads sold by Invitrogen as part of their “The Dynabeads mRNA DIRECT™ Kit”, where oligo-(dT)₂₅ residues are covalently linked to the Dynabeads. In a standard reaction, approximately 2 μg of polyadenylated RNA is bound to 250 μl of Dynabeads Oligo (dT)₂₅ following the directions of the manufacturer.

### Example 8

### Fill-in Reaction Using a Reverse Transcriptase

A fill-in reaction on a surface is preferably done in the presence of trehalose to increase the efficiency of the M-MLV reverse transcriptase in the reaction. The reaction can be performed in solution or using immobilized RNA bound to a surface by hybridization between a polyA tail of the RNA and an oligo-dT oligonucleotide attached to the surface. The oligo-dT oligonucleotide primers the fill-in reaction, which is restricted by adding only dTTP to the reaction mixture:

| RNA/cDNA hybrid on solid support | x | μl slurry |
|---|---|---|
| 5x Buffer | 30 | μl |
| 10 mM dTTP | 4 | μl |
| Saturated Sorbitol/Trehalose | 30 | μl |
| M-MLV (200 U/μl) | 15 | μl |
| Add water to a final volume | 150 | μl |

The reaction can be conducted in a thermocyler using the following settings:
25° C., 30 sec
42° C., 30 min
50° C., 10 min
56° C., 10 min
60° C., 10 min
Hold at 4° C. until further use.

The reaction is terminated by Proteinase K treatment by adding:

| | |
|---|---|
| Proteinase K | 3 μl |
| 0.5M EDTA | 6 μl |

After incubation at 45° C. for 20 min, the Proteinase K can be removed by washing the beads with a washing buffer following the maker's direction. If conducted in solution, the RNA can be further purified by CTAB precipitation:

| | |
|---|---|
| CTAB/urea (1% CTAB/4M urea) | 300 μl |
| 5M NaCl | 30 μl |

After incubation at room temperature for 10 min, the RNA is collected by centrifugation at 15000 rpm and mom temperature for 15 min. The pellet can be completely dissolved in 200 μl of 7M guanidine-HCl, and the RNA is further purified by ethanol precipitation with the addition of 500 μl ethanol, following the direction given in the previous examples. Finally the RNA pellet is dissolved in 50 μl water.

### Example 9

### Reverse Transcription Reaction to Synthesis cDNA Template

Similar reaction conditions as given in the previous example are used to synthesize a cDNA strand having a sequence complementary to a RNA template, if all 4 nucleotides are offered in the reaction mixture. The reaction can be primed by an oligo-dT primer or can make use of a primer mixture containing primers of random sequence or defined sequence. Again the reaction may be conducted in solution, or it can be conducted using RNA templates immobilized on a solid support. In brief, for example 20 μg of RNA and 5.6 μg of an oligo-dT primer or 4.8 μg of random primer were mixed in a total volume of 22 μl. After incubation at 65° C. for 10 min, the sample is chilled on ice for 2 min. In another tube, an enzyme mixture is prepared containing 30 μl of 5 times buffer, 4 μl of 10 mM dNTP, 30 μL of saturated Sorbitol/Treharose, and 3,000 units of M-MLV reverse transcriptase in a total volume of 128 μl. To start the reverse transcription reaction, the RNA sample and the enzyme mixture are combined, and the reaction is conducted on a thermocycler set at: 25° C. for 30 sec, 42° C. for 30 min, 50° C. for 10 min, 56° C. for 10 min, 60° C. for 10 min, and held at 4° C. The reaction is terminated by adding 3 μl of proteinase K and 6 μl of 0.5 M EDTA and incubation at 45° C. for 20 min. For reactions in solution, the RNA can be purified by CTAB precipitation with the addition of 300 μl of 1% CTAB/4M urea and 30 μl of 5 M NaCl. After incubating at room temperature for 10 min, the precipitate is harvested by centrifugation at 15,000 rpm and room temperature for 15 min. The pellet was dissolved in 200 μl of 7 M guanidine HCl and further purified by adding 500 μl ethanol. After precipitation and washing under the conditions described in the previous examples, the RNA is finally dissolved in 50 μl water.

### Example 10

### RNase Treatment and Read Out of 5′ Ends of Full-Length mRNAs on Solid Support

Treating DNA/RNA hybrids with RNase I will remove all regions made of single-stranded RNA. This reaction can be performed in solution or on DNA/RNA hybrids immobilized on a solid support. In a standard reaction, the RNase I is added to a slurry:

| | | |
|---|---|---|
| RNA/cDNA solution | 180 | μl |
| 10x RNaseI buffer | 20 | μl |
| 10 u/μl RNaseI | 2 | μl |

After incubation at 37° C. for 30 min, the RNase can be destroyed by Proteinase K treatment:

| | |
|---|---|
| Proteinase K | 2 μl |
| 0.5M EDTA | 4 μl |

The mixture is incubated at 45° C. for 30 min. For reaction conducted on a surface, remaining DNA/RNA hybrids are washed with a washing buffer according to the maker's directions. If the reaction is conducted in solution, the DNA/RNA hybrids can be further purified by removing the Proteinase K by phenol/chloroform extraction followed by isopropanol precipitation under the conditions given in a previous example. When working with small amounts of DNA/RNA hybrids, it can be advisable to add tRNA to increase the yields of the precipitation as given in the example below:

| | | |
|---|---|---|
| Sample | 250 | μl |
| 20 μg/μl tRNA | 1 | μl |
| 5M NaCl | 12.5 | μl |
| Isopropanol | 250 | μl |

After the washing steps the DNA/RNA hybrids can be dissolved in 50 μl of 0.1×TE buffer.

### Example 11

### Removal of RNA Molecules by Alkali Treatment

The RNA portion within DNA/RNA hybrids can be removed by alkali treatment of the DNA/RNA hybrids. The reaction can be performed in solution or for DNA/RNA hybrids bound to a solid support. If the DNA portion within the DNA/RNA hybrids is attached to the solid support, the DNA will remain attached to the support as single-stranded DNA molecules. In a standard reaction, the slurry is incubated with:

| | | |
|---|---|---|
| cDNA/RNA hybrids | 50 | μl slurry |
| 50 mM NaOH/5 mM EDTA | 300 | μl |

The reaction mixture is incubated for 5 min at room temperature, and the alkali buffer is quickly replaced by 100 μl 1M Tris-HCl pH7.0 buffer. It is advisable not to leave the DNA/RNA hybrids for an extended time with the alkali buffer, and after the alkali treatment, the remaining alkali has to be washed away carefully. To increase the efficiency of the RNA removal step, RNase I can be added to the Tris buffer. An example for a standard reaction in solution includes:

| | | |
|---|---|---|
| DNA in 1 M Tris HCl, pH 7.0 | 100 | μl |
| 10 unit/μl RNaseI | 1 | μl |

After mixing the DNA with the RNase I the reaction mixture is incubated at 37° C. for 10 min. The RNase I can be removed by adding 2 μl of Proteinase K and 7 μl of 10% SDS. In brief, the reaction mixture is incubated at 45° C. for 15 min before extraction with equal volumes of phenol/chloroform and chloroform. For isopropanol precipitation, 3 μl of 1 μg/μl glycogen, 22.5 μl of 5 M NaCl and 450 μl of isopropanol are added. After precipitation at −20° C. for at least 30 min, the precipitate is collected by centrifugation at 15,000 rpm and 4° C. for 30 min, washed first with 800 μl of 80% ethanol and second time with 100 μl of 80% ethanol, before the pellet is finally dissolved in 50 μl of 0.1×TE buffer.

### Example 12

### G-Tailing Reaction

The 3′ end of cDNA can be modified in a G-tailing reaction to introduce a polyG homopolymer that can be used as a priming site for the 2^{nd} strand cDNA synthesis. The reaction can be conducted in solution or for cDNA bound to a solid support. Different amounts of dGTP are used where the ratio between the dGTP concentration and the cDNA amount should be kept in a proper range to restrict the length of the polyG homopolymer. For example, for a standard reaction, the following ratios have been used successfully:
20 μM dGTP for >750 ng cDNA
15 μM dGTP for >400 ng cDNA
10 μM dGTP for >150 ng cDNA
5 μM dGTP for <150 ng cDNA

To conduct the reaction, the cDNA is heated to 65° C. for 2 min to destroy secondary structures that may interfere with the reaction. Thereafter, the following components are added:

| 50 μM dGTP in saturated trehalose | x | μl |
|---|---|---|
| 5x TdT buffer | 10 | μl |
| 10 mM MgCl2 | 5 | μl |
| Terminal deoxynucleotidyl transferase (Takara) | y | μl (40 Units) |
| Add water to a final volume of | 50 | μl |

The reaction mixture is incubated for 15 min at 45° C., before it is terminated by adding 1 μl of 0.5 M EDTA. In case the reaction is conducted in solution, the terminal deoxynucleotidyl transferase can be removed by Proteinase K treatment as described in the previous examples. For reaction performed on a solid support, remaining terminal deoxynucleotidyl transferase is removed by washing with a suitable washing buffer following the maker's directions, though the Proteinase K treatment can also be applied to immobilized cDNA. Proteinase K treatment can be preferable over conducting washing steps only, in which an enzyme associates with the substrate.

### Example 13

### Single-Stranded Linker Ligation Reaction

More preferable than the addition of a homopolymer to the 3′ end of cDNA, a specific linker can be added to the 3′ end of the cDNA in a ligation reaction. Such linker may have special features as outlined in the description of the invention. A single-stranded linker ligation reaction can be carried out in solution or using cDNA immobilized on a solid support. In a standard reaction, the cDNA is incubated with a double-stranded linker having an overhang made of single-stranded DNA. Such a linker can be prepared as described by Shibata et al. in Biotechniques June 2001; 30(6):1250-1254. The reaction is setup by mixing:

| cDNA (up to I μg) | x | μl |
|---|---|---|
| Linker (2 μg) | y | μl |
| Add 0.1x TE to a final volume of | 7.5 | μl |
| TAKARA Ligation kit Sol. II | 7.5 | μl |
| TAKARA Ligation kit Sol. I | 15 | μl |

After incubating at 16° C. overnight, the reaction is terminated. For reactions conducted in solution, the ligase can be removed by Proteinase K with the addition of 10 μl of 0.1×TE buffer, 1 μl of 0.5 M EDTA, 1 μl of Proteinase K and 1 μl of 10% SDS. In brief, the reaction mixture is incubated at 45° C. for 15 min before extraction with equal volumes of phenol/chloroform and chloroform. For isopropanol precipitation 3 μl of 1 μg/μL glycogen, 5 μl of 5 M NaCl and 100 μl of isopropanol are added. After precipitation at −20° C. for at least 30 min, the precipitate is collected by centrifugation at 15,000 rpm and 4° C. for 30 min, washed first with 800 μl of 80% ethanol and second time with 100 μl of 80% ethanol, before the pellet is finally dissolved in 50 μl of 0.1×TE buffer.

### Example 14

### Exaction of Sequence Information from Reads

Sequencing reads obtained from the sequencer can be analyzed for sequences derived from adaptor sequences or primers. Since the sequences of all adapters and primers used in the experiment are known, sequencing reads can be aligned to adapter and primer sequences by standard software solutions, such as NCBI BLAST (http://www.ncbi.nlm.nih.gov/BLAST/), so as to align sequence reads to genomic sequences. Sequences derived from adaptors and primers are removed and excluded from further analysis. Where available, quality scores for each base calling event may be considered to remove low quality sequences from the data analysis.

### Example 15

### Mapping of Sequencing Reads to Genome

Sequence information obtained by means of the present invention can be used to identify transcribed regions within genomes for which partial or entire sequence information has been obtained. Most commonly, mapping experiments align sequences from sequencing reads with genomic sequences in databases. Such a mapping can be performed using standard software solutions, such as NCBI BLAST (http://www.ncbi.nlm.nih.gov/BLAST/), so as to align sequence reads to genomic sequences.

### Example 16

### Computational Identification of Transcript Start Sites

5′ end specific sequences that are mapped to genomic sequences allow for the identification of Transcript Start Sites and regulatory sequences around the Transcript Start Sites. In genomes the regions upstream of the 5′ end of transcribed regions usually encompasses most of the regulatory elements which are used in the control of gene expression. Other regulatory sequences may also be found downstream of the Transcript Start Sites. Regulatory sequences can be further analyzed for their functionality by searches in databases which hold information on binding sites for transcription factors. Publicly available databases on transcription factor binding sites and for promoter analysis include, for example:

Transcription Regulatory Region Database (TRRD) (http://wwwmgs.bionet.nsc.ru/mgs/dbases/trrd4/)
TRANSFAC (http://www.gen-regulation.com/index.html)
TFSEARCH (http://www.cbrc.jp/research/db/TFSEARCH.html)
PromoterInspector provide by Genomatix Software (http://www.genomatix.de/)

Alternative views on promoter regions may be taken by defining a window size covering the number of nucleotides upstream and downstream of the Transcript Start Sites to be included in the analysis.

### Example 17

### Alignment of Sequences Against Each Other to Identify RNA-RNA Interactions

Sequences obtained from the same plurality of RNA molecules within a sample or as retrieved from genomic regions can be analyzed in an alignment experiment by a standard software solution like NCBI BLAST (http://www.ncbi.nlm.nih.gov/BLAST/) to identify RNA molecules or regions of complementary sequence.

### Example 18

### Preparation of an RNA Molecule for Use as an Internal Control

A RNA molecule or a group of RNA molecules can be prepared in an in vitro transcription reaction using a DNA dependent RNA polymerase, namely T3 or T7 RNA polymerase, and a template having the appropriate promoter for the DNA dependent RNA polymerase at the S′ end of the cDNA insert in the following setup:

| | | |
|---|---|---|
| DNA template in TE buffer (about 3 μg of DNA) | 10 | μl |
| 5 times reaction buffer | 40 | μl |
| 0.1M DTT | 20 | μl |
| 10 mg/ml BSA | 1.6 | μl |
| 10 mM rNTP | 10 | μl |
| RNase free H₂0 | 115.4 | μl |
| T7 or T3RNA polymerase (20 U/μl) | 3 | μl |
| Total reaction volume | 200 | μl |

The reaction mixture is incubated at 37° C. for 5 hours prior to terminating the reaction. Remaining DNA template is destroyed by DNase treatment in the following setup:

| | | |
|---|---|---|
| Reaction mixture from previous reaction | 200 | μl |
| 10 mM CaCl₂ | 20 | μl |
| RNase free DNase I (1 U/μl) | 1 | μl |

The reaction mixture is incubated at 37° C. for 1 hour prior to terminating the reaction. Remaining enzyme activities within the reaction mixture can be removed by treatment with Proteinase K. The RNA may be further purified by standard steps, such as phenol/chloroform extraction for removing proteins, and ethanol precipitation for changing the salt concentration. Otherwise the RNA may be purified by use of a commercial product such as the QIAGEN RNeasy purification kit.

The quality of the RNA can be analyzed by the ratios of the OD readings at 230, 260 and 280 nm to monitor the RNA purity. An effective removal of proteins is achieved when the 260/280 ratio was higher than 1.8 or around 2.0. The RNA samples can further be analyzed by electrophoresis in an agarose gel or the use of an Agilent 2100 bioanalyzer to confirm the correct length of the RNA molecules.

What is claimed is:

1. A method for simultaneous identification and analysis of RNA molecules in a sample, comprising the steps of:
preparing a solid support which has capturing oligonucleotide molecules attached onto its surface,
introducing a functional group at the 3′ end of RNA molecules present in the sample,
binding the RNA molecules by hybridization to capturing oligonucleotide molecules which have a sequence complementary to the sequences of the functional group and which are fixed on a solid support, and
subjecting the RNA molecules to analysis as attached to the solid support.

2. The method according to claim 1, wherein the analysis is direct sequencing of the RNA molecules.

3. The method according to claim 1, where the analysis includes priming reverse transcription of at least some of the RNA molecules fixed to the solid support to obtain cDNA sequences complementary to the RNA molecules, washing away the RNA molecules so as to obtain single-stranded cDNA, and subjecting the cDNA to sequencing while it remains attached to the solid support.

4. A method for simultaneous identification and analysis of RNA molecules in a sample, comprising the steps of:
preparing a solid support which has capturing oligonucleotide molecules attached onto its surface,
introducing a functional group at the 3′ end of RNA molecules present in the sample,
labeling one or more diol groups present in the RNA molecules with a labeling molecule,
binding the RNA molecules by hybridization to the capturing oligonucleotide molecules which have a sequence complementary to the sequences of the functional group,
detecting the labeling molecule introduced to the RNA molecules to identify a feature, including existence or absence of a Cap structure, of each RNA molecule attached to the solid support, and
subjecting the RNA molecules to analysis as attached to the solid support.

5. The method according to claim 4, wherein the analysis includes priming reverse transcription of at least some of the RNA molecules fixed to the solid support to obtain cDNA sequences complementary to the RNA molecules, and washing away the RNA molecules so as to obtain single-stranded cDNA, and subjecting the cDNA to sequencing while it remains attached to the solid support.

6. A method for simultaneous identification and analysis of mRNA molecules in a sample, comprising the steps of:
preparing a solid support which has capturing oligonucleotide molecules attached onto its surface,
labeling one or more diol groups present in the mRNA molecules with a labeling molecule,
binding the mRNA molecules to the capturing oligonucleotide molecules which have a sequence complementary to a sequence of the 3′ end of the RNA molecules,
detecting the labeling molecules attached to the mRNA molecules to identify a feature, including existence or absence of a Cap structure, of each mRNA molecule attached to the solid support, and
subjecting the mRNA molecules to analysis as attached to the solid support.

7. A method for simultaneous identification and analysis of mRNA molecules in a sample, comprising the steps of:
preparing a solid support which has capturing oligonucleotide molecules attached onto its surface,
labeling the mRNA molecules with a suitable labeling molecule,
binding the mRNA molecules by hybridization to the capturing oligonucleotide molecules, which have a sequence complementary to the sequence of the functional group introduced at the 3′ end of the RNA molecules,
detecting two labeling molecules for a single mRNA molecule so as to determine that said single RNA molecule is full length having a labeled Cap structure and a labeled polyA tail, and
subjecting the full-length mRNA to analysis as attached to the solid support.

8. A method for simultaneous identification and analysis of mRNA molecules in a sample, comprising the steps of:
preparing a solid support which has capturing oligonucleotide molecules attached onto its surface,
labeling one or more diol groups present in the mRNA molecules with a suitable labeling molecule,
binding the mRNA molecules by hybridization to the capturing oligonucleotide molecules, which have a sequence complementary to the sequence of the functional group introduced at the 3′ end of the RNA molecules,
priming reverse transcription of the mRNA molecules attached to the solid support to obtain cDNA strands complementary to the mRNA molecules so as to form DNA-RNA hybrids,
subjecting the DNA-RNA hybrids to RNase I treatment for removal of their single stranded portion,
detecting the labeling molecule to identify any cDNA strands in the hybrids that have reached the 5′ end of any full-length mRNA,
washing away the RNA molecules so as to obtain single-stranded cDNA, and
adding a priming site to the single-stranded cDNA molecules which represent full-length mRNA to analysis for sequencing of the 3′ end of such cDNA while it remains attached to the solid support.

9. A method for simultaneous identification and analysis of RNA molecules in a sample obtained from a single cell or tissue, comprising the steps of:
preparing a solid support which has capturing oligo-dT oligonucleotide molecules attached onto its surface,
modifying RNA molecules in the sample at their 3′ end so as to introduce a functional group in a polyadenylation reaction,
extending the RNA molecules having a polyA tail at the 3′ end by adding a polyC tail to their 3′ end,
labeling diol groups of RNA molecules within the sample by introducing a fluorescent dye,
binding the labeled RNA molecules to a solid support by binding of the functional group to the capturing oligo-dT oligonucleotide molecules on the solid support,
detecting labeled RNA molecules on the solid support to determine loading of the solid support and a percentage of capped mRNA molecules within the sample,
having the capturing oligonucleotide priming a reverse transcription reaction to synthesize cDNA strands attached to the solid support,
removing, by RNase I digestion, labels from 3′ ends of RNA molecules and from Cap structures of DNA-mRNA hybrids that have not extended to reach the 5′ end of mRNA templates,
detecting full-length cDNAs for capped mRNAs on the solid support to determine a percentage of capped mRNA or full-length cDNA molecules in the sample,
removing the RNA templates,
ligating a double-stranded adapter with a priming site to the 3′ end of the single-stranded cDNA attached to the solid support, and
driving, using the priming site within the adapter, a sequence reaction to obtain sequence information from the 5′ end of RNA molecules in the sample as the cDNA molecules remain attached to the solid support.

10. A method for simultaneous identification and analysis of RNA molecules in a sample obtained from a single cell or tissue, comprising the steps of:
preparing a solid support which has capturing oligo-dT oligonucleotide molecules attached onto its surface,
modifying RNA molecules in the sample at their 3′ end so as to introduce a functional group in a polyadenylation reaction,
optionally extending the RNA molecules having a polyA tail at the 3′ end by adding a polyC tail to their 3′ end,
labeling diol groups of the RNA molecules by introducing a fluorescent dye,
binding the labeled RNA molecules to a solid support by binding of the functional group to the capturing oligo-dT oligonucleotide molecules on the solid support
detecting labeled RNA molecules on the solid support to determine loading of the solid support and a percentage of capped mRNA molecules in the sample,
having the capturing oligonucleotide prime a reverse transcription reaction to synthesize cDNA strands attached to the solid support and having a sequence complementary to the polyA tail of the RNA template bound to the capturing oligonucleotide, and
having the extended capturing oligonucleotide prime a reverse transcription reaction to obtain sequence information from the 3′ end of RNA molecules in the sample as the cDNA strands remain attached to the solid support.

11. A method for simultaneous identification and analysis of RNA molecules in a sample obtained from a single cell or tissue, comprising the steps of:
preparing a solid support which has capturing oligo-dT oligonucleotide molecules attached onto its surface,
modifying RNA molecules in the sample at their 3′ end so as to introduce a functional group in a polyadenylation reaction,
extending RNA molecules having a polyA tail at the 3′ end by adding a polyC tail to their 3′ end.
labeling diol groups of the RNA molecules by introducing a fluorescent dye.
binding the labeled RNA molecules to a solid support by binding of the functional group to the capturing oligo-dT oligonucleotide molecules on the solid support,
detecting labeled RNA molecules on the solid support to determine loading of the solid support and a percentage of capped mRNAs within the sample,
having the capturing oligonucleotide prime a reverse transcription reaction to synthesize cDNA molecules attached to the solid support and having a sequence complementary to the polyA tail within the RNA template bound to the capturing oligonucleotide.
having the extended capturing oligonucleotide prime a reverse transcription reaction to obtain sequence information from the 3′ end of RNA molecules within the sample.
having the extended capturing oligonucleotide prime a reverse transcription reaction to synthesize cDNA strands attached to the solid support and having a sequence complementary to the RNA template bound to the capturing oligonucleotide,
removing, by RNase I digestion, labels from 3′ ends of RNA molecules and from Cap structures of cDNA-mRNA hybrids that have not extended to reach the 5′ end of mRNA templates,
detecting full-length cDNAs for capped mRNAs on the solid support to determine a percentage of capped mRNAs or full-length cDNAs within the sample.
removing RNA templates,
ligating a double-stranded adapter with a priming site to the 3′ end of the single-stranded cDNA strand attached to the solid support, and
driving, using the priming site within the adapter, a sequence reaction to obtain sequence information from the 5′ end of RNA molecules in the sample while the cDNA strands remain attached to the solid support.

12. A method for simultaneous identification and analysis of RNA molecules in a sample obtained from a single cell or tissue, comprising the steps of:
preparing a solid support which has capturing oligonucleotide molecules attached onto its surface,
incubating total RNA molecules contained in the sample with the solid support,
having polyadenylated mRNA molecules within the sample hybridize to the capturing oligonucleotide molecules on the solid support while nonpolyadenylated RNA molecules do not bind,
washing away RNA molecules that do not bind to the solid support,
labeling diol groups in mRNA molecules on the solid support by introducing a fluorescent dye,
detecting the labeled mRNA molecules on the solid support to determine loading of the solid support and a percentage of capped mRNAs in the sample,
having the capturing oligonucleotide prime a reverse transcription reaction to synthesize cDNA strands attached to the solid support and having a sequence complementary to the polyA tail of the mRNA templates attached to the solid support,
having the extended capturing oligonucleotide prime a reverse transcription reaction to obtain sequence information from the 3′ end of mRNA molecules attached to the solid support,
having the extended capturing oligonucleotide prime a reverse transcription reaction to synthesize cDNA strands attached to the solid support and having a sequence complementary to the mRNA templates attached to the solid support,
removing, by RNase I digestion, labels from 3′ ends of mRNA molecules and from Cap structures of cDNA-mRNA hybrids that have not extended to reach the 5′ end of the mRNA templates,
detecting full-length cDNAs for capped mRNAs on the solid support to determine a percentage of capped mRNAs or full-length cDNAs in the sample,
removing the RNA templates,
ligating a double-stranded adapter with a priming site to the 3′ end of the single-stranded cDNA attached to the solid support, and
driving, using the priming site within the adapter, a sequence reaction to obtain sequence information from the 5′ end of mRNA molecules in the sample while the cDNA strands remain attached to the solid support.

13. A method for obtaining sequence information from a DNA molecule in a sample in a cyclic reaction comprising the steps of:
adding to the sample an adapter having a priming site to a 3′ end of a DNA molecule and a recognition site for a restriction endonuclease that cuts outside of its binding site,
hybridizing a sequencing primer to the primer site to drive a sequencing reaction,
obtaining sequence information from the 3′ end of the DNA molecule,
removing a DNA fragment from the 3′ end of the DNA molecule by digestion with the restriction endonuclease that cuts outside of its binding site,
adding a new adapter having a priming site and a recognition site for a restriction endonuclease that cuts outside of its binding site to the end of the DNA molecule opened by the removing step, and
repeating the above steps.

14. A method for detecting RNA molecules having one or more diol groups, comprising the steps of:
introducing a labeling molecule to one or more diol groups of an RNA molecule so as to obtain labeled RNA,
adding the labeled RNA to a sample, and
detecting the labeling molecule for identifying the RNA molecule in the sample.


SEQUENCE LISTING (5 sequences)

SEQ ID NO: 1 - DNA, 16 bp, Artificial Sequence
  Description of Artificial Sequence Synthetic oligonucleotide
aaaaaaaaaaaaaaaa

SEQ ID NO: 2 - DNA, 20 bp, Artificial Sequence
  Description of Artificial Sequence Synthetic oligonucleotide
tttttttttttttttttttt

SEQ ID NO: 3 - DNA, 19 bp, Artificial Sequence
  Description of Artificial Sequence Synthetic oligonucleotide
ttttttttttttttttttt

SEQ ID NO: 4 - DNA, 10 bp, Artificial Sequence
  Description of Artificial Sequence Synthetic oligonucleotide
tttttttttt

SEQ ID NO: 5 - DNA, 25 bp, Artificial Sequence
  Description of Artificial Sequence Synthetic oligonucleotide
ttttttttttttttttttttttttt